(19) United States
(12) Patent Application Publication
(10) Pub. No.: US 2006/0052661 A1
(43) Pub. Date: Mar. 9, 2006

(54) Minimally invasive control surgical system with feedback

(75) Inventors: Israel Gannot, Ramat Hasharon (IL); Alon Goren, Moshav Ben-Shemen (IL); Abraham Dayan, Bat Yam (IL)

Correspondence Address: Martin Moynihan;Anthony Castorina, 2001 Jefferson Davis Highway, Suite 207, Arlington, VA, 22202, US

(73) Assignee: Ramot At Tel Aviv University Ltd., Tel Aviv (IL)

(21) Appl. No.: 10/542,926

(22) PCT Filed: Jan. 22, 2004
(86) PCT No.: PCT/IL04/00067

§ 371 Date: Jul. 21, 2005

(30) Foreign Application Priority Data
Jan. 23, 2003 (IL) 154101

(51) Int. Cl. A61B 18/18; A61B 1/04; A61B 6/00; A61B 1/06

(52) U.S. Cl. 600/108; 600/160; 600/473; 606/11

(57) ABSTRACT

In therapeutic endoscopy micro-knives, electrical diathermy or laser beam replace the conventional hand-held scalpel. The laser “knife” has substantial capabilities over the rest: it can cut incisions, coagulate hemorrhages and ablate (vaporize) neoplastic tissues, while interaction is contact-less and sterile. The laser energy is delivered transendoscopically through rigid or flexible fiberoptic waveguides. Flexible endoscopes are used to access body curved spaces and cavities, such as the digestive tract. A control system for minimally invasive surgery, and more particularly, for laser surgery using infrared laser is provided. A feedback mechanism is provided to obtain thermographic information from the targeted site and a processor uses this thermographic information to monitor and control input parameters, including air flow, suction, and laser beam parameters. Furthermore, an infrared imaging fiber bundle is used in combination with an infrared camera to provide the thermographic information to the processor. Specifically, the system and methods provided can be used to more effectively present very specific wavelengths of laser treatment, with capability of monitoring its effects and altering parameters at the time of treatment. Furthermore, means for thermographic analysis of the targeted area, wherein such analysis provides a guideline for the monitoring and altering of the controllable parameters is provided.

### FIELD AND BACKGROUND OF THE INVENTION

The present invention relates to a system and method for real-time control of minimally invasive procedures using flexible laser waveguides with thermal mapping feedback. More particularly, the present invention relates to a system and method for using flexible laser waveguides in the mid-infrared range to treat sensitive conditions via minimally invasive surgery, and for using thermal feedback, all within a central control system which is able to update and change variable parameters during real-time processing.

Minimally Invasive Surgery (MIS) has revolutionized the practice of many surgical disciplines, replacing many conventional procedures. MIS focuses on minimizing invasiveness by utilizing technological innovation in video imaging, optical waveguiding and endoscopical instrumentation. These techniques require very small incisions, if any, thus limiting the disturbance of healthy tissue and minimizing psychological trauma as compared to conventional surgery. The decreased pain, discomfort and blood loss, the shorter postoperative hospitalization and faster return to daily activities and consequently the reduced direct and indirect costs, are all factors in the acceptance of this method.

It is possible for surgeons to neither look directly at nor touch the tissues or organs on which they operate, but rather to view them through an endoscope. An endoscope is a tube-like device, inserted through body natural orifices or 1-2 cm punctures, and maneuvered to the region of interest. They enable the surgeon to screen, diagnose and repair body organs internally. The functions performed by MIS include obtaining biopsies, coagulating incisions, ablating cancerous cells and polyps and cutting tissues. Surgical micro-tools make it possible to grasp, suck, clip, hold and cut endoscopically, but their overall size is miniature, which limits the amount that can be done. The advent of lasers, refinements to diathermy, shock wave generators, ultrasound devices and hydraulic devices have extended the surgical capabilities (e.g. coagulation, ablation). The mechanical actions are an extension of conventional surgical work on a small scale at a distance.

Gastroscopy, also known as upper endoscopy, gastrointestinal (GI) endoscopy, panendoscopy or esophagogastroduodenoscopy (EGD), lets the surgeon examine and treat gastroscopically the lining of the upper part of the gastrointestinal tract, which includes the esophagus, the stomach and the duodenum. Gastroscopy helps in evaluating symptoms of persistent upper abdominal pain, nausea, vomiting or difficulty swallowing. It enables minimal invasive diagnosis of bleeding from the upper GI tract, and is more accurate than X-ray imaging for detecting inflammation, ulcers and tumors of the esophagus, stomach and duodenum. It is also used to obtain biopsies in order to distinguish between benign and malignant tissues or to test for Helicobacter pylori (bacterium that causes ulcers). In addition, upper endoscopy is performed when a cytology test is needed for cell analysis.

One of the primary therapeutic means in gastroenteroscopy is the laser. Gastroenterologists were the first to use lasers in conjunction with flexible endoscopes. Today, Nd:YAG lasers are used routinely and considered effective in controlling hemorrhage (via coagulation), treating benign, malignant or non-neoplastic stenosis such as peptic ulcers and vascular anomalies, and recanalization of dysphagia (obstructing cancers of the upper GI tract). However, the Nd:YAG laser at a wavelength of 1.064 μm does not provide optimum radiation for all GI diseases.

Stomach Cancer and Barrett's Esophagus

Stomach cancer and Barrett's Esophagus are two examples of GI diseases which are not readily treatable using a standard Nd:YAG laser. Treatment for stomach cancer is generally prompt removal of the tumor by surgery. This may require removing part or all of the stomach, by either conventional surgery or MIS, via micro-knives, diathermy, ablative lasers or photodynamic therapy (PDT). If the stomach cancer has started to spread, removing affected parts of neighboring organs is needed as well. If all of the cancer present in the body cannot be removed by surgery, chemotherapy may be given. Radiotherapy plays a limited role in the treatment of stomach cancer since radiation doses strong enough to destroy these cancer cells could seriously damage the surrounding healthy tissue. Thus, treating gastric cancer in its very first stage is essential. However, when early cancer spreads superficially, thin slices of mucosa layers needed to be ablated. None of these techniques has definitively demonstrated high specificity, yet efficacy, in doing so.

Barrett's esophagus is defined by the metaplasia of existing squamous mucosa into specialized intestinal-type mucosa. Thus, the esophageal lining changes, and becomes similar to the mucosa that lines the intestine. This condition is associated with the development of adenocarcinoma of the esophagus. Barrett's tissue is visible during endoscopy, although a diagnosis by endoscopic appearance alone is not sufficient. The definitive diagnosis of Barrett's esophagus requires biopsy confirmation.

Elimination of the metaplastic mucosa may decrease the cancer risk. Currently, several forms of therapy have evolved with the goal of replacing the specialized mucosa with normal squamous mucosa. These proposed treatments include PDT and thermal techniques. The effectiveness of PDT varies depending on the pharmaceutical photosensitizer used and the wavelength of light applied to activate the drug. Thermal techniques include multipolar coagulation, argon plasma coagulation, KTP YAG laser therapy, Nd:YAG laser therapy, and argon laser therapy. Mucosal resection has been attempted through the endoscope to remove large areas of the Barrett mucosa. All of these ablative strategies attempt to destroy the metaplastic mucosa, and promote the re-growth of squamous epithelium. These therapies have demonstrated the ability to “reverse” the metaplasia to varying degrees, but a decrease in cancer risk has not been demonstrated conclusively with any of these treatment methods.

Other conditions which do not seem to benefit from application of conventional laser therapies via endoscopy may include any other superficial cancer. Furthermore, any lesion within the body that can be accessed via existing openings or small incisions have the potential to benefit from the present invention.

Laser Types

Laser energy is emitted as a narrow collimated beam of very high intensity, which, when used transendoscopically, provides a means of delivering energy to precise sites inside the body with increased precision, improved homeostasis, less tissue manipulation and mechanical trauma, and greater ease of sterility maintenance as compared with conventional surgical techniques.

Medical applications make use of three fundamental mechanisms of laser-tissue interactions: photochemical, photothermal and photomechanical. The biological effects of laser energy depend on the laser wavelength, the irradiance, the exposure duration, and the opto-thermal properties of the tissue involved. Surgical procedures that involve coagulation or ablation of tissue are thermal. In this interaction, laser light is partially absorbed by tissue and converted into heat energy, which raises tissue temperature. Initially, this causes thermal contraction of the treated area. As tissue shrinks, small vessels are sealed, which can stop hemorrhage, and thus cause coagulation. Higher energies, such as those used for ablation, kill the cells in situ and ultimately vaporize cellular material.

The types of laser currently in widespread use for their photothermal effects are: CO₂ (10.6 μm) and Er:Yag (Erbium Yttrium Aluminum Garnet, 2.94 μm) in the mid infrared range (IR), Nd (Neodymium) YAG (1064 nm) in the near IR and Argon ion, which has 2 main lines (488 and 514 nm) in the blue and green regions of the visible spectrum. The CO₂ and the Er:YAG laser beams are strongly absorbed in water, and hence in soft tissue as well, which is comprised 70-90% of water). The other two types of laser are absorbed more in pigmented cells. Er:YAG lasers are used to evaporate both soft and hard tissues.

The differences between these wavelengths manifest themselves in the extent of damage of surrounding areas. In soft tissue, such as the bladder, applying sufficient energy to vaporize solely the surface cells causes lesser damage to a depth of only 0.1 mm with the CO₂ laser. When the same surface effect is produced with the Argon laser, mild damage extends for 1 mm, and with the Nd:YAG, it extends for 5 mm.

The volume heated by the CO₂ laser outside the area of vaporized tissue is so small that it has little practical value for stopping major hemorrhage, although it can seal capillary oozing. In contrast, the highly localized effect of this laser makes it eminently suitable as a laser knife, the cells immediately under the beam being vaporized with minimal damage to adjacent areas. This beam can cut through tissue or scan across the surface of an organ to vaporize the superficial layers to any desired depth, a characteristic which could be particularly beneficial in the treatment of superficial carcinomas such as Barrett esophagus and mucosal gastric cancer. However, neither CO₂ nor Er:YAG laser beam have been transmitted in a suitable medium for use in treatment of such conditions via minimally invasive techniques.

CO₂ lasers have mainly two modes of operation: continuous wave (CW) and pulsatile wave (PW). In the CW mode, the laser delivers the same amount of energy for as long as the laser is activated. This mode releases the highest average power. However, a byproduct is the formation of a zone of thermal coagulum at the cut edge, which can impede the biological healing process.

PW mode reduces this thermal side effect, by minimizing the heat conduction effect, during which heat is transferred from the irradiated volume to adjacent areas within the tissue. This transfer is managed by transmitting pulse trains or “Superpulses” where each pulse lasts a milli- or micro-second. During ablative therapy, the short-energetic pulses enable instant evaporation of matter, such that heat conduction is further minimized. The technique of Q-switched laser enables extremely high power output in the megawatt range over a very short time in the nanosecond range. Thus, PW modes can minimize the damage zone, minimize the surgical duration, and deliver ablative energy within every pulse, so that cells will vaporize instantly before heating the surrounding tissue.

Two classes of pulsed lasers are candidates for ablation in medical applications: the UV laser (λ<300 nm) and the mid-IR laser (λ>2.0 μm). The UV light is highly absorbed by lipids, proteins, and nucleic acids whereas mid-IR light (2.94 μm and 10.6 μm) is strongly absorbed in water. The UV laser has the potential to precisely ablate tissue, leaving little thermal damage, but it may cause significant mechanical damage to the tissue and is potentially mutagenic. Mid-IR lasers are easy to maintain, compact, and less expensive, yet may produce mechanical and thermal damage to the adjacent tissue.

The clinical success of PW treatment is often limited by the extent of damage that is caused to the tissue in the vicinity of the ablation crater. In general, pulsed ablation seems to be a trade off between thermal damage to the surrounding tissue, caused by relatively long pulses (>1100 ms), and mechanical damage to surrounding tissue, caused by relatively short pulses (<1 ms).

CO₂ lasers have a major role in therapy applied through rigid endoscopes such as those used in gynecology, otolaryngology and airway obstruction surgeries. They are also used in genitourinary, plastic, dental, hepatic, orthopedic, and cardiovascular surgery and are considered the mainstay of laser neurosurgery. Since IR emittance is invisible, CO₂ laser is combined with low power, helium-neon (He—Ne) or diode laser-pointer. However, applications of CO₂ lasers are limited to “straight lines.” Their beams are maneuvered with a series of articulated mirrors (within the machine's arms), and straight rigid waveguides (used for rigid endoscopes).

Factors Affecting Peripheral Tissue Damage

The effect of laser treatment on peripheral tissue can be measured by the optical and thermal penetration depths. When a laser beam irradiates a tissue, part of its energy is reflected from the surface. The rest of the laser energy penetrates into inner layers, where it is absorbed and scattered by the tissue.

The term optical penetration depth denotes the depth within which scattering and absorption in a biological tissue attenuates an incident laser beam. Within this depth, the beam energy can induce significant biological effects. The optical penetration depth is highly dependent on the wavelength of the incident laser radiation. For thermal ablation of tissue, peak absorption of 2.94 μm and high absorption at 10.6 μm are preferred.

The thermal penetration depth is generally defined as the distance in which the temperature has decreased to 1/e of its peak value.

Laser-induced heating has biological implications, resulting in tissue alterations, which can be characterized as follows:
1. 37-42° C.: local warming only—no biological alternations
2. 42-50° C.: hyperthermia: structural changes of molecules, accompanied by bond destruction and membrane alterations of the cells; edema is observed; after long exposure time (several minutes), necrosis may start.
3. 50-60° C.: reduction in enzyme activity, cellular energy transfer and repair mechanisms (i.e. repair of mistakes in DNA producing); inflammation may occur.
4. 60° C.: permanent denaturation of proteins and collagen leads to tissue coagulation and shrinkage (generally in blood vessels); necrosis with healing by regeneration
5. 80-100° C.: permeabilization of the membrane is drastically increased, destroying the chemical concentration equilibrium; necrosis with healing by scarring or later sloughing
6. 100° C.: water molecules, contained in most tissues, start to vaporize; cavitation bubbles cause ruptures and thermal decomposition of tissue fragments, whereby adjacent tissue is preserved.
7. 150° C.: carbonization; observable by the blackening of tissue and smoke escaping.
8. 3000° C. and up: melting

The extent of thermal tissue damage is dependent on a combination of tissue temperature and duration of temperature, as illustrated in the graph of FIG. 1.

Ablation of soft tissues via high-energy laser pulses may also generate mechanical side effects, such as shock waves and cavitations. A cavitation bubble can expand and collapse several times within a matter of microseconds. When this occurs near a solid boundary, a high-speed liquid jet is produced, which results in an asymmetric collapse. If the bubble contacts the solid during its collapse, the jet can cause a high impact pressure against the solid wall.

Thus, various factors can affect surrounding tissue, including laser type, duration of laser application, and atmospheric conditions.

Thermography

Measurement of thermal effects on tissue can be accomplished via thermography, which makes use of the infrared spectral band to determine temperature changes. At the short-wavelength end the boundary lies at the limit of visual perception, in the deep red zone. At the long-wavelength end it merges with the microwave radio wavelengths, in the millimeter range. The IR band is often further subdivided into four smaller bands, the boundaries of which are also arbitrarily chosen. They include: the near IR (0.75-2 μm), the mid IR (2-20 μm), the far and the extreme IR (20-100 μm).

A blackbody is defined as an object which absorbs all radiation that impinges on it at any wavelength. The radiation characteristics of an aperture in an isotherm cavity made of an opaque absorbing material represents almost exactly the properties of a blackbody. By providing such an isothermal cavity with a suitable heater it becomes what is termed a ‘cavity radiator’. An isothermal cavity heated to a uniform temperature generates blackbody radiation, the characteristics of which are determined solely by the temperature of the cavity. Such cavity radiators are commonly used as sources of radiation in temperature reference standards in the laboratory for calibrating thermographic instruments, such as an infrared (IR) camera.

If the temperature of blackbody radiation exceeds 525° C., the source begins to be visible so that it no longer appears black to the eye. This is the incipient red heat temperature of the radiator, which then becomes orange or yellow as the temperature increases further. In fact, the definition of the so-called ‘color temperature’ of an object is the temperature to which a blackbody would have to be heated to have the same appearance.

Infrared Imaging (IRI) in Medicine

Diagnosis of thermal damage and/or pathology is a primary goal in all types of medical procedures. IRI has been used since the early 1970s for medical applications. Abnormalities such as tumor angiogenesis, malignancies, inflammation, and infection cause localized increases in temperature, which appear as hot spots or as asymmetrical patterns in an infrared thermogram.

While the use of IRI has increased constantly in industrial and security applications, until recent years it has declined in medicine, probably because of limitations in the first generation of IR cameras. The earlier instrumentation was not adequately sensitive to detect the subtle changes in temperature needed to accurately detect and monitor disease. Furthermore, since the skin temperature distribution can result from a variety of internal and external conditions that affect the circulation and metabolism, IRI has been considered a non-specific diagnostic technique.

In recent years the military has greatly improved the spatial and the thermal resolutions of JR cameras to a level approaching 0.001° C., a level at which 0.5 to 1° C. differences or changes in temperature can be reliably measured. The development of Focal Plane Array (FPA) IR sensors, for a range of wavelengths incorporating 9 to 9.5 μm (where Planck function peaks for body temperature), has allowed high quality imaging as well as the export of digital temperature measurements amiable to computer-assisted image analysis. Today, libraries of image processing routines are available to analyze images captured both statically and dynamically. Some of these analytical tools are aimed at resolving the problem of non-specificity.

The medical community has only recently acquired the new state-of-the-art FPA IRI equipment and has begun to investigate its medical utilities. In-vivo studies have endorsed its clinical value in many disciplines: breast cancer (risk assessment, detection, prognosis and therapeutic monitoring), burn trauma (staging), diabetes, cardiology (vulnerable plaque), neurology, urology, dermatology, ophthalmology, neonatology, pain management and anxiety detection. Fewer clinical studies have reported the use of IRI of internal tissue surfaces intraoperatively. Neurosurgeons have exhibited the IRI capability to detect vascular occlusion and reperfusion as well as its potential to explore cerebrovascular disease, epilepsy, functional cortical activation, and brain tumors.

IRI and Thermograhy in Minimally Invasive Procedures

Unlike direct IR measurements, utilizing IRI by minimally invasive means is a greater challenge, as the IR images need to be transferred from the internal tissue surface to the IR camera transendoscopically. The investigation of IRI and/or thermocouple-based thermography during MIS has just recently begun. Several studies—from a variety of medical disciplines—that have used experimental apparatuses are briefly described below.

Ogan et al. in “Infrared thermography and thermocouple mapping of radiofrequency renal ablation to assess treatment adequacy and ablation margins,” Urology, 2003, 62(1): p. 146-151 have exploited translaparoscopic IRI to monitor the surface renal temperature during RF ablation. Tests done on female pigs resulted in a correlation between thermocouple measurements during RF ablation and thermographic findings post mortem, demonstrating the feasibility of using IRI for detection of lethal temperatures. In-vivo coronary bypass MIS in beagles has been carried out using IRI by Nakagawa et al., as described in “Intraoperative thermal artery imaging of an EC-IC bypass in beagles with infrared camera with detectable wave-length band of 7-14 microm: Possibilities as novel blood flow monitoring system,” Minimally Invasive Neurosurgery, 2003, 46(4): p. 231-234. Nakagawa et al. concluded that transendoscopic IRI (7-14 μm) may provide a non-invasive functional angiography. Stefanadis et al. in “Increased temperature of malignant urinary bladder tumors in vivo: the application of a new method based on a catheter technique,” Journal of Clinical Oncology, 2001, 19(3): p. 676-681, have developed a thermocouple-based thermography catheter, and demonstrated in a human in-vivo trial that thermal gradients between healthy and neoplastic tissue zones could be a useful criterion in the diagnosis of malignancy in tumors of the human urinary bladder. They found significant temperature differences (e.g. 1° C.) between patients with benign and malignant tumors. The endodontists McCullagh et al. compared thermocouple-based thermography versus IRI of temperature rise on the in-vitro root surface during the continuous wave of condensation technique, as described in McCullagh et al, “A comparison of thermocouple and infrared thermographic analysis of temperature rise on the root surface during the continuous wave of condensation technique,” International Endodontic Journal, 2000, 33(4): p. 326-332. They found IRI to be a useful tool for mapping temperature change over a large area. In 2001, Cadeddu et al. reported nine laparoscopic urologic procedures in patients using IRI (3-51 μm) via a rigid endoscope, as described in Cadeddu et al, “Laparoscopic infrared imaging,” Journal of Endourology, 2001, 15(1): p. 111-116. They stated that IRI proved to be useful in differentiating between blood vessels and other anatomic structures, and that in contrast to conventional endoscopy, vessel identification, assessment of organ perfusion, and transperitoneal localization of the ureter were successful in all instances.

Naghavi et al. in “First prototype of a 4-French, 180-degree, side-viewing infrared fiberoptic catheter for thermal imaging of atherosclerotic plaque,” American Journal of Cardiology, 2001, 88(2A): p. 81E-81E, reported the first prototype of a flexible infrared fiberoptic catheter for the IRI of atherosclerotic plaques. They built a 1.5 m long hexagonal bundle of 19×100 μm Chalcogenide fibers, and a ZnSe mirror assembled to its distal tip to obtain side-IR-viewing. The catheter was connected to an FPA IR camera. The system had a thermal resolution of 0.01° C. and a spatial resolution of 100 μm. Temperature heterogeneity was successfully detected in a phantom model, simulating blood vessels and hot plaques, as well as in an animal in-vivo study. They concluded that the technique is feasible and can be used for thermal detection of vulnerable atherosclerotic plaques. U.S. Pat. No. 5,445,157 to Adachi et al. describes a thermographic endoscope having an insert part that is inserted into a body cavity. The thermographic endoscope includes an infrared image forming device disposed in the distal end of the insert part so as to form an infrared image of a part under inspection, and a device for transmitting the infrared image formed by the infrared image forming device to the outside of the insert part. The thermographic endoscope further includes a device for converting the infrared image, which is transmitted by the infrared image transmitting device, into a visible image and for displaying the visible image, and a device for injecting low-temperature gas outwardly from the distal-end of the insert part of the endoscope.

Feedback and Control

The complexity of the various parameters related to tissue damage when using laser therapies leads to a situation in which the ability to control these parameters would be highly advantageous. Furthermore, a mechanism for thermographic feedback which could be incorporated within such a control system would provide a further advantage of providing an assessment of the various controllable parameters within a minimally invasive surgical session. None of the prior art systems include means for delivering laser beams in the mid-IR, or suitable means for thermographic analysis during real-time procedures. Furthermore, none of the prior art systems provide the capability to control various parameters relating to the procedure during real-time assessment via thermographic analysis.

There is thus a widely recognized need for, and it would be highly advantageous to have, a system for providing and on-line monitoring of a laser in the mid-IR range with thermal feedback for treatment of specific disorders, as will be described in greater detail hereinbelow.

### SUMMARY OF THE INVENTION

According to one aspect of the present invention, there is provided, a system for monitoring and control of a minimally invasive surgical procedure for a targeted tissue. The system includes an endoscope having an input conduit for receiving an input for the targeted tissue and an output conduit for sending output from the targeted tissue, an input mechanism for providing input to the targeted tissue through the input conduit of the endoscope, a thermal output receiver for receiving thermal information from the targeted tissue through the output conduit of the endoscope, and a processor in communication with the input mechanism and the thermal output receiver, the processor configured to receive the thermal output from the thermal output receiver and to adjust the input mechanism so as to adjust the input to the targeted tissue based on the thermal output, and wherein the receiving and adjusting is performable during a real-time procedure.

According to another aspect of the invention, there is provided a device for introduction of an infrared laser to a target tissue. The device includes an endoscope having a conduit and a flexible hollow wave guide placed within the conduit, wherein the flexible hollow waveguide includes a hollow tube, a metal layer on the inner surface of the hollow tube, and a thin dielectric film of silver iodide over the metal layer.

According to yet another aspect of the present invention, there is provided a system for monitoring and control of thermal properties of tissue during a minimally invasive surgical procedure. The system includes an endoscope having a laser delivery conduit with a flexible hollow waveguide for delivery of an infrared laser to the tissue, and an imaging conduit having an infrared imaging fiber bundle placed within the imaging conduit for reading thermal properties of the tissue, a laser generator for providing an infrared laser to the tissue through the laser delivery conduit of the endoscope, an infrared camera for receiving thermal information from the targeted tissue through the imaging conduit of the endoscope, and a processor in communication with the laser generator and the infrared camera, the processor configured to receive the thermal information from the infrared camera and to adjust the laser generator so as to adjust parameters of the laser provided to the tissue based on the thermal information, and wherein the receiving and adjusting is performable during a real time procedure.

According to yet another aspect of the present invention, there is provided a system for monitoring of minimally invasive surgery at a targeted site. The system includes a device for surgical contact with the targeted site, having a distal end and a proximal end, wherein the distal end is configured to contact the targeted site and the proximal end is accessible to a user, and an opening at the distal end connected to an opening at the proximal end, an infrared imaging fiber bundle having a distal end and a proximal end, the infrared imaging fiber bundle positioned within the device such that the distal end of the infrared imaging fiber bundle is located at the opening of the distal end of the device, and the proximal end of the infrared imaging fiber bundle is located at the opening of the proximal end of the device, wherein the distal end of the infrared imaging fiber bundle is configured to receive output information from the targeted site, and the proximal end of the infrared imaging fiber bundle is configured to send the output information to an output device, and an output device in communication with the proximal end of the infrared imaging fiber bundle.

According to yet another aspect of the present invention, there is provided a method for monitoring and control of a minimally invasive surgical procedure. The method includes introducing an infrared laser beam to a surgical site, the introducing including using adjustable parameters, measuring thermal properties of the surgical site in response to the introducing, processing the measured thermal properties, and adjusting the adjustable parameters based on the processing, wherein the adjusting is done during a time frame of the minimally invasive surgical procedure, and wherein the steps can be repeated as many times as necessary so as to achieve acceptable thermal properties.

According to further features in preferred embodiments of the invention described below, the endoscope can be a gastroscope, laparoscope, arthroscope, cystoscope, ureteroscope, pharyngoscope, bronchoscope, nephroscope or any other suitable device. In preferred embodiments, the input conduit includes a laser beam conduit and the input is a laser beam. The system can further include a flexible hollow waveguide placed within the input conduit, and wherein the flexible hollow waveguide is suitable for receiving an infrared laser beam therethrough. In preferred embodiments, the input conduit includes several input conduits, including a gas insufflation conduit, a suction conduit, a surgical tool conduit, and an illumination conduit. In a preferred embodiment, the output conduit is a viewing conduit, and more specifically an infrared viewing conduit. An infrared imaging fiber bundle may be placed within the infrared viewing conduit for infrared thermal imaging of the tissue. In a preferred embodiment, the thermal output receiver is an infrared thermal output receiver, and more specifically is an infrared camera.

According to further features in preferred embodiments of the invention described below, the processor includes a controller for controlling the input based on the thermal output. The processor may further include a thermal reader for reading the thermal output and incorporating the thermal output into a usable format, such as a map or a database.

According to further features in preferred embodiments of the invention described below, the device further includes an infrared laser generator in communication with the endoscope for generating an infrared laser beam through the flexible hollow waveguide to the target tissue. The device further includes a feedback device in communication with the target tissue, the feedback device configured to receive output information from the target tissue upon application of the infrared laser beam. In a preferred embodiment, the feedback device is a thermal receiver and the output information is thermal information from the target tissue. The thermal receiver preferably includes an infrared imaging fiber bundle in communication with the camera, wherein the infrared imaging fiber bundle is positioned through an additional conduit in the endoscope.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graphical illustration of thermal effects on surrounding tissue;

FIG. 2 is a block diagram illustration of a system for control and monitoring of minimally invasive surgery, in accordance with a preferred embodiment of the present invention;

FIGS. 3A and 3B are perspective and cross-sectional illustrations of an endoscope for use in a preferred embodiment of the present invention;

FIGS. 4A and 4B are cross-sectional illustrations of a hollow waveguide in accordance with an embodiment of the present invention;

FIG. 5 is an illustration of a flexible hollow waveguide, in accordance with a preferred embodiment of the present invention;

FIG. 6 is a laser beam delivery system, in accordance with an embodiment of the present invention;

FIG. 7 is an adaptor assembly from the laser beam delivery system of FIG. 6 in greater detail;

FIG. 8 is an illustration of a portion of the laser beam delivery system of FIG. 6;

FIGS. 9 and 10 are illustrations of a hollow laser beam waveguide of the laser beam delivery system of FIG. 6;

FIG. 11 is a graphical illustration of the relationship between laser power, beam diameter, and average power density.

FIG. 12 is a block diagram illustration of and adjustable laser system in accordance with an embodiment of the present invention;

FIG. 13 is a block diagram illustration of an insufflating system in accordance with an embodiment of the present invention;

FIG. 14 is a block diagram illustration of a surgical suction system in accordance with an embodiment of the present invention;

FIG. 15 is an illustration of the electromagnetic spectrum, showing the range of detection for thermal imaging;

FIG. 16 is a graphical illustration of Planck's formula;

FIG. 17 is a cross-sectional illustration of an infrared imaging fiber bundle in accordance with a preferred embodiment of the present invention;

FIG. 18 is a schematic illustration of a camera and thermographic measurement;

FIG. 19 is a schematic illustration of an infrared imaging fiber bundle and an infrared camera, and their relative setup;

FIG. 20 is an illustration of a Labview main panel for control, data acquisition, and optimization;

FIG. 21 is a graphical illustration of the effects of varying airflow, suction and wave type on temperature changes, in accordance with an example illustrated in the present application;

FIG. 22 is a graphical illustration of the effect of suction on temperature changes, in accordance with an example illustrated in the present application;

FIG. 23 is a graphical illustration of the effects of varying power on temperature changes, in accordance with an example illustrated in the present application; and

FIGS. 24-27 are graphical illustrations of the effects of suction and CO₂ flow on temperature changes, in accordance with an example illustrated in the present application.

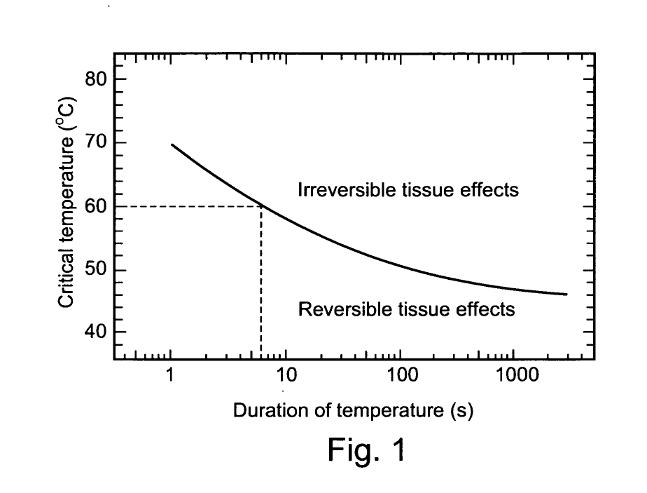

### DESCRIPTION OF THE PREFERRED EMBODIMENTS

The present invention is of a system and method for control and on-line monitoring of a laser for minimally invasive treatment of specific disorders. Specifically, the present invention can be used to more effectively present very specific wavelengths of laser treatment, with capability of monitoring its effects and altering parameters at the time of treatment. Furthermore, the present invention provides means for thermographic analysis of the targeted area, wherein such analysis provides a guideline for the monitoring and altering of the controllable parameters.

The principles and operation of a system and method according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

I. Overview of System

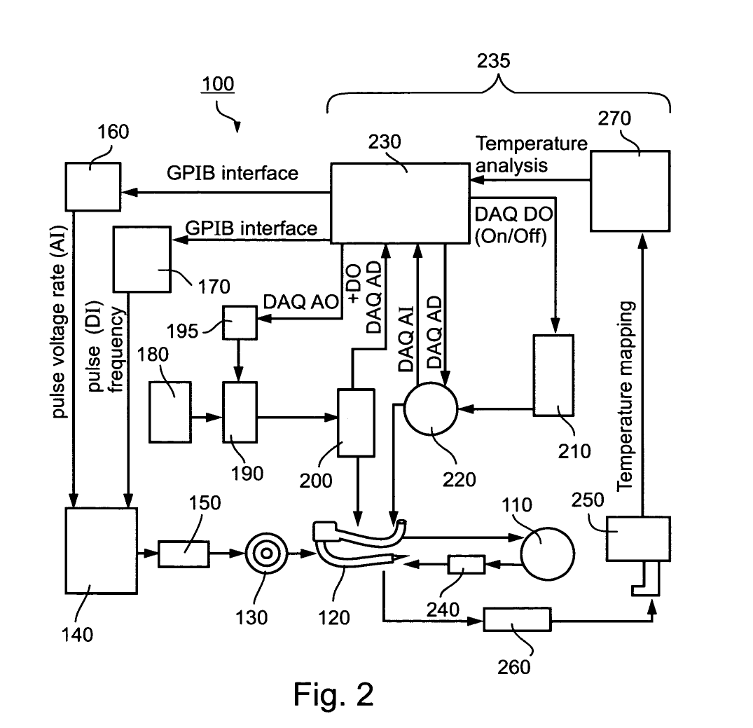

Reference is now made to FIG. 2, which is a block diagram illustration of a system 100, in accordance with a preferred embodiment of the present invention. System 100 includes a target tissue 110 to which will be applied a laser treatment and from which measurements will be taken. Application of such laser treatment is provided by an endoscope 120, which is fitted with a hollow waveguide 130 for delivery of a laser beam. Hollow waveguide 130 is preferably flexible so that it can be administered through a flexible endoscope 120, thus providing ease of delivery and minimal invasiveness. The laser beam is produced in a laser generator 140 and sent through hollow waveguide 130 via an optic coupler 150. In a preferred embodiment, laser generator 140 is a CO₂ laser beam generator, for producing CO₂ lasers in the mid-IR range. However it should be noted that any suitable laser may be generated and would fall within the scope of the present invention.

Parameters of the laser beam are controllable via a power supply 160 and a function generator 170. Endoscope 120 is further provided with means for receiving a supply of gas and suction. Gas supply is provided by a gas insufflator 180, which in a preferred embodiment comprises CO₂ gas for viewing and cooling of the site. Gas insufflator 180 sends the gas through a proportional valve 190, for control and regulation of gas flow, preferably by computer-controlled voltage, and via a pressure and flow meter 200 through endoscope 120 for delivery to target tissue 110. Endoscope 120 has control mechanisms for controlling the amount of gas which can flow to target tissue 110. Suction is provided by a standard surgical suction provider 210, and is delivered via a vacuum control and meter 220. All three parameters which run through endoscope 120, that is, laser beam, gas supply, and surgical suction, are in turn controllable via a controller 230. Controller 230 may be incorporated within a processor 235 as software, hardware, or any combination thereof. In a preferred embodiment, controller 230 includes a software setup such as a Labview application (National Instruments, USA), although any suitable program may be used.

Thermographic measurements of target tissue 110 are taken via an infrared imaging fiber bundle 240, which interacts with target tissue 110, and sends thermal information to an infrared camera 250 via an optical coupler 260. In an alternative embodiment, direct measurements may be taken from infrared camera 250 without the use of fiber bundles. Infrared camera 250 provides a temperature map via a thermographic analyzer 270. Thermographic analyzer 270 can be included within processor 235 or may be separate from processor 235. Furthermore, thermographic analyzer 270 may comprise software, hardware, or any combination thereof. In a preferred embodiment, thermographic analyzer 270 comprises a thermal control system such as ThermaCAM researcher (FLIR Systems, Sweden), which is capable of both translating input temperature data into a map and providing a temperature analysis. Data from thermographic analyzer 270 is provided to controller 230, which can, in turn, use the provided information to control the laser beam, gas supply, and surgical suction based on the temperature analysis.

Individual components of system 100 will be described in detail in the sections to follow.

II. Presentation of Laser Beam to Surgical Site

Endoscope

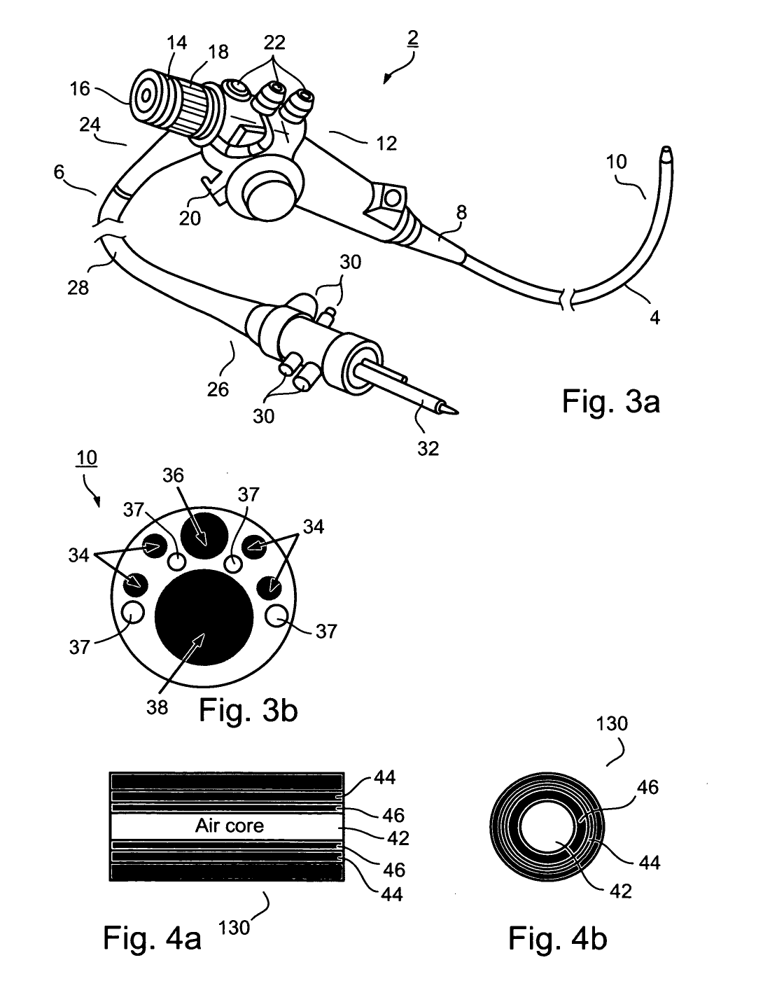

Reference is now made to FIGS. 3A and 3B, which are perspective and cross-sectional illustrations of an endoscope 2 for use in a preferred embodiment of the present invention. Endoscope 2 can be an adapted gastroscope, laparoscope, arthroscope, cystoscope, uretetoscope, pharyngoscope, bronchoscope, nephroscope, or any other device used for minimally invasive procedures. Endoscope 2 includes a first flexible shaft 4 and a second flexible shaft 6. First flexible shaft 4 is an insertion tube and is locatable within a body cavity. First flexible shaft 4 has a proximal end 8 and a distal end 10. A more detailed view of distal end 10 is illustrated in the cross-sectional view of FIG. 3B, described in further detail hereinbelow. Proximal end 8 is connected to an instrument control section 12, through which a physician can guide distal end 10 and control fluid flow in and out of the body cavity. Instrument control section 12 may vary, but generally includes a camera mount 14 with an eye piece 16, for viewing of the body cavity, a diopter adjustment ring 18 for adjusting the viewing capacity, a control knob 20 for controlling an angle of distal end 10, and a series of valves 22 for adjusting gas, suction, air, water and any other adjustable parameters. Second flexible shaft 6 has a distal end 24 and a proximal end 26. Distal end 24 is connected to instrument control section 12 by a universal cord 28. Proximal end 26 includes connectors 30 for gas, water, suction and for a light guide 32 for introduction of a laser beam.

Reference is now made to FIG. 3B, which is a cross-sectional illustration of distal end 10 of first flexible shaft 4, in accordance with a preferred embodiment of the present invention. Distal end 10 includes illumination fibers 34, a visible light viewing fiber 36, and an infrared viewing fiber 38. Additionally, a number of open channels 37 are provided for flow, suction and surgical instrumentation. Illumination fibers 34 are fibers that help illuminate the surgical area, so that the site can be properly viewed. Visible light viewing fiber 36 includes a visible coherent bundle having a few tens of thousands of single fibers, which permits a user to view the surgical site via camera mount 14. Infrared viewing fiber 38 is a specialized infrared imaging fiber, which will be described in further detail hereinbelow. Surgical micro-tools, which are able to grasp, suck, clip, hold and cut, can be introduced through at least one of open channels 37. Furthermore, laser-transmitting waveguides can be introduced through open channels 37 as well.

It should be readily apparent that the embodiment illustrated in FIGS. 3A and 3B is merely exemplary, and that any commercially available endoscope or adapted minimally invasive device may be used in preferred embodiments of the present invention.

Waveguide

In order to utilize the laser advantageously within body cavities, the beam must be delivered via a waveguide, threaded through, for example, light guide 32 of an endoscope such as endoscope 2. Flexible endoscopes entail flexible waveguides, which should meet the following requirements:
i. There should be minimal energy loss to avoid damage of the endoscope or tissue;
ii. The output beam should be as close as possible to a smooth Gaussian beam, in which most of the energy is centralized;
iii. Fiber substances should be biocompatible; and
iv. Fabrication must be repeatable.
   For surgical applications the optimal choice for delivery of a mid-IR laser beam is a hollow waveguide, as such waveguides are able to transmit high peak laser power (megawatts) and up to 100 watts of continuous wave radiation with losses of less than 1 dB/m.

Reference is now made to FIGS. 4A and 4B, which are cross-sectional illustrations of a hollow waveguide 130. Hollow waveguide 130 includes a hollow tube 42, internally coated with a metal layer 44 and a thin dielectric layer 46. Optical radiation is guided through reflection and refraction from metal layer 44 and dielectric layer 46.

Hollow waveguides are characterized by the guiding mechanism of the radiation. The main attenuation mechanisms are reflection from thin dielectric layer 46, and scattering due to surface roughness. Reflection and refraction determine the wavelength to be transmitted and its attenuation, whereas scattering changes the ray's propagation angle and energy distribution, causing losses.

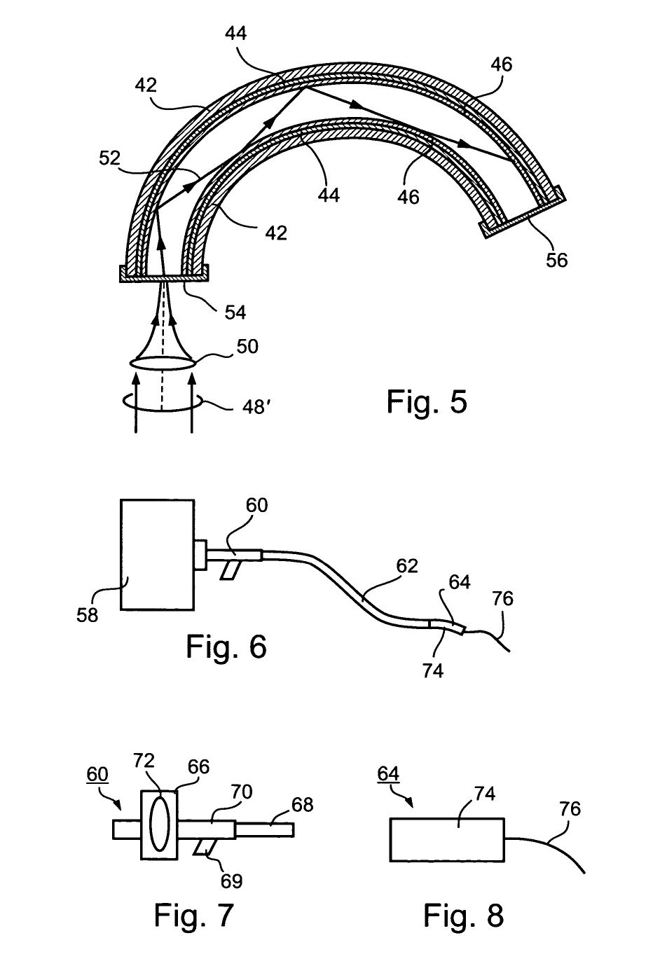

Flexible hollow waveguides for use within the mid-IR range have been developed. An example of a flexible hollow waveguide useful within the mid-IR range is described, for example, in U.S. Pat. No. 4,930,863 to Croitoriu et al. Reference is now made to FIG. 5, which is an illustration of the hollow waveguide described in the above-referenced application.

The drawing diagrammatically illustrates a hollow fiber waveguide for transmitting radiation energy in the carbon dioxide laser having a wavelength of 10.6 μm. The hollow fiber waveguide comprises a hollow, flexible, plastic tube 42, a metal layer 44 on the inner surface of tube 42, and a dielectric layer 46 on metal layer 44.

As also shown in the drawing such a hollow fiber waveguide may be used for transmitting a laser beam, schematically indicated at 48. The beam, after focusing by lens 50, is directed into one end of the waveguide and is transmitted through the waveguide by internal reflection as shown by the arrows 52, to the opposite end of the waveguide, even though the waveguide is bent to a relatively small radius of curvature. The ends of the waveguide may be closed by transparent windows or films or lenses 54, 56, to prevent the entry of dirt. The windows or films or lenses 54, 56 may be thin transparent plastic film welded over the end of the tube, or a transparent inorganic material e.g. ZnSe glued to the tube.

Another possibility is to use a conical type optical element or a lens 50 at the inlet of the fiber waveguide to direct the laser beam into the fiber waveguide; and at the outlet to use a similar conical element or lens for refocusing the beam and thereby to increase the energy density at the outlet.

Such a hollow fiber waveguide is particularly useful for transmitting the beam of a carbon dioxide laser or other type of electromagnetic radiation to a desired location, e.g., for surgical, communication or for material processing applications.

In one preferred example, the hollow plastic tube 42 may be of flexible polyethylene having an internal diameter of 3 mm, an external diameter of 6 mm. Other types of plastic materials could be used, including polypropylene, polystyrene, fluoropolymers, polyamides (e.g., nylon 6, nylon 11), polyurethanes, natural or synthetic rubber, silicone rubber and polyvinyl chloride. The dimensions of the hollow tube can be varied; also its cross-section can have different geometrical shapes besides the circular shape illustrated, including square, rectangular or ellipsoidal.

Metal layer 44 applied on the inner surface of hollow plastic tube 42 is preferably of silver, but could be of other metals, including nickel, copper, gold and palladium. Its thickness is preferably up to 10 microns.

Following are several examples for producing the hollow fiber waveguide according to the invention.

### EXAMPLE 1

The hollow plastic tube 42 is of polyethylene having an internal diameter of 3 mm, an outer diameter of 6 mm, and a length of 700 mm.

Its inner surface is first etched with a solution of sulphochromic acid at room temperature for a period of 20 minutes; alternatively, a mixture of sulphochromic acid and phosphoric acid at a temperature of 500 degrees Celsius may be used, in which case the etching period is reduced to about 3 minutes.

Following the application of the etching agent, the inner surface of hollow plastic tube 42 is then rinsed with distilled or deionized water at room temperature for one minute.

The inner surface of hollow plastic tube 42 is then subjected to dilute hydrochloric acid at room temperature for about 3 minutes, to effect acidification and Cr⁶ neutralization. The inner surface is then subjected to a sensitizing agent comprising a solution of SnCl₂/HCl at room temperature for 5 minutes; rinsed with distilled or deionized water at room temperature for 1 minute; then subjected to an activating agent comprising a solution of PdCl₂/HCl at room temperature for 3 minutes; and then again rinsed with distilled or deionized water at room temperature for 1 minute.

A mixture of a silver-plating solution and a reducer solution is then circulated through the hollow plastic tube in contact with its inner surface. Following is one example of the silver-plating solution, and of the reducer solution which can be used.

Silver-Plating Solution

The following materials are dissolved in small amounts of distilled or deionized water: 5 gr AgNO₃, plus 30 mg sodium dodecylbenzenesulfonate, plus 25 ml of 28% ammonia solution. To this solution is added pure or diluted acetic acid so that the final pH is reduced to 6-9. After the pH adjustment, the final volume of the solution is increased to 100 ml by adding distilled or deionized water. The silver concentration is Ca 0.3 moles/dm³.

Concentrations of Ca 0.1-1.0 moles/dm³ silver could generally be used, with the understanding that the ammonia/acetic acid concentration and the reducer concentration would be adjusted accordingly.

It is possible to add additives by dissolving water-soluble polymeric materials (e.g., polyvinyl pirrolidone; polyethylene glycoles; polyacrylic acid); these additives tend to improve the adhesion of the silver layer to the polymeric substrate. The silver-plating solution is stable and can be stored for up to a week without significant loss of activity.

Reducer Solution

The reducer solution is prepared by diluting a 3 molar stock solution of N₂H₄:H₂O (hydrazine hydrate) down to 0.3 moles/dm³ solution. Other reducers that may be used include inverted sugars or formaldehyde.

25 ml of the above silver-plating solution are mixed with 6 ml of the above reducer solution, and the mixture is diluted to 100 ml. The reducer solution is added in small amounts to the silver-plate solution over a period of 5-10 minutes. The mixture is brought into contact with the inner surface of hollow plastic tube 42 at a temperature of about room temperature or up to 30 degrees Celsius for 5-10 minutes. The transfer rates of the solution into the hollow plastic tube are approximately 5 mm/min. The deposition initially takes place on the activated inner surface of the hollow plastic tube, making the process purely electroless.

Following the deposition of metal layer 44 on the inner surface of hollow plastic tube 42, the tube is rinsed with distilled or deionized water at room temperature for 1 minute.

The dielectric layer 46 is then deposited on the inner surface of the metal layer 44. This is done by subjecting the inner surface of the silver coating into contact for a few seconds at room temperature with a water solution containing 10% (W/W) polyvinyl pirrolidone and 0.1-0.5% (W/W) iodine.

The interior of hollow plastic tube 42 is then rinsed with distilled or deionized water at room temperature for a few seconds, or with carbontetrachloride. Following the rinsing, the transparent windows 54, 56 are applied by welding polyethylene film to the ends of the tube, or gluing an inorganic transparent window or lens to the tube.

The above process produces a hollow fiber waveguide having an outer diameter of 6 mm, an inner diameter of 3 mm, a metallic layer of 2-microns, and a dielectric layer of 10 microns. Such a hollow fiber is very flexible and capable of transmitting the laser beam of a carbon dioxide laser having a wavelength of 10.6 micrometers. The energy transmission through the waveguide is dependent on its length, radius of curvature, and the location of the focus of the beam. The transmission decreases with the decreasing radius of curvature, reaching an almost constant value of 30-35% at a radius of curvature of 150 mm, with a tube length of 500 mm.

An additional example of making flexible hollow waveguides, in accordance with the present invention follows:

### EXAMPLE 2

A flexible plastic tube is used made of fluoropolymers known by their trade names: Teflon, PTFE (Poly-Tetra-Fluoro-Ethylene), FEP (Fluorinated-Ethylene-Propylene), PFA (Per-Fluoro-Alkoxy). Other types of plastic tubes may also be used, e.g., polyethylene, polypropylene, nylon 6, nylon 11, silicone rubber, rubber, P.V.C., and polystyrene. The length of the tube in this example is 1.2 meters; the inner diameter (ID) is 2.4 mm; and the outer diameter (OD) 3.2 mm. The ID and OD values may vary between fractions of mms and up to tens of mms. The tube is processed as follows:

1. Stretching and straightening: The tube (sold in rolls) is cut into the appropriate length, and is then subjected to stretching by pulling it in a vertical form with a 3-5 kg weight for a few hours.

2. Etching I: The etching solution is a Na Naphthalene solution in Tetrahydrofuran with a concentration range of 0.8-1.8 moles/dm³ (preferably 1.2 moles/dm³). The inner walls of the tubes are brought into contact with the solution for periods of 0.5-5 minutes, depending on the solution age and concentration. The tube is then washed with tetrahydrofuran, acetone and deionized water (DIW).

3. Etching II: The inner walls of the tube are brought into contact with a sulfochromic acid solution for half a minute at room temperature, washed with DIW, subjected to diluted hydrochloric acid (HCl) for 3 minutes, and washed again with DIW.

4. Sensitization: The inner surface of the tube is brought into contact with SnCl₂/HCl solution (SnCl₂-70 gr/dm³, HCl-40 gr of HCl (con)/dm³) for 5-20 minutes at room temperature and then rinsed with DIW.

5. Activation: The inner surface of the tube is reacted with a PdCl₂/HCl solution (1 gr/dm³ of PdCl₂, 5 ml/dm³ of HCl (con) for 5-20 minutes at room temperature and then rinsed (very thoroughly) with DIW. Other known post activation methods, e.g., rinsing with HCl, NaOH or E.D.T.A. solutions, may also be used.

6. Silver plating: The now activated inner surface of the tube is brought into contact with a silver-plating solution and a reducer solution which are prepared as follows:

Silver-Plating Solution: The following materials are dissolved (in this order) in a small amount of water: 5 gr AgNO₃, 30 mg of dodecyl benzen sulfonic acid sodium salt, 30 ml of 28% ammonia solution. To the resulting solution, pure or diluted organic or inorganic acids are added (preferably acetic acid) so that the final pH is reduced to 7.5-10. The volume of the solution is then increased to 100 ml by adding DIW (Ag⁺ concentration is Ca 0.3 moles/dm³). It is also possible to dissolve the silver salt and buffer solution in other organic polar solvents as dimethylsulfoxide, ethylene glycol, hexamethylene phosphoramide, or mixtures of such polar solvents with water in various proportions. Concentrations of Ca 0.1-1.2 moles/dm³ Ag⁺ may generally be used, in which case the ammonia, acid and reducer concentrations should be changed accordingly. The silver solution is filtered through a filter-paper 24 hours after preparation and only then is it ready for use. The silver solution is very stable and insensitive to light and can be stored even in stoppered clear glassware for months without loss of activity.

Reducer Solution: This solution is prepared by diluting a 3 moles/dm³ stock solution of hydrazine hydrate (NH₂HN₂—H₂O) to Ca 0.3 moles/dm³ solution; other reducers, e.g., inverted sugars or formaldehyde, may also be used.

Silver Plating Process: 25 ml of the silver-plating solution is diluted to 50 ml by DIW. Next, 10 ml of the reducer solution are diluted to 20 ml and added very slowly over a period of 10-20 minutes to the silver solution with constant stirring. The solution should be in contact with the inner walls of the tube (or tubes) all the time. This may be done in room temperature. The deposition initially takes place only on the activated tube surface and thus makes the reaction purely “electroless”.

The above described solution may be used to plate up to 50 cm² of plastic surface. Several of the surface preparation solutions may be recycled. The silver plated tubes are rinsed with DIW and dried in a stream of inert gas (N₂) for 20 minutes.

Dielectric layer preparation: The dried, silver plated tube is brought into contact with a halogen element solution (e.g., 0.0125-7% (W/W), but preferably 1.25% (W/W) I₂ crystals dissolved in CCl₄ or the same concentration of Br₂) for 0.05-10 minutes (preferably 1-2 minutes) and is then washed consecutively with CCl₄ and acetone, and dried in a stream of inert gas (N₂) for 20 minutes to produce a silver halide.

The above stated process is used to produce a hollow fiber waveguide with a metallic layer of up to 2 μm thickness and a dielectric layer of up to 2 μm thickness. The waveguide is very flexible and capable of transmitting CO₂ laser energy wavelength (wavelength=10.6 μm); energy transmission depends on the length, radius of the tube, radius of curvature and the coupling mode. The transmission for a 0.7 meter long fiber of OD=3.2 mm, ID=2.4 mm is 80% when straight, and decreases to 60% when the radius of curvature is 14 cm. This means that the influence of the radius of curvature on the transmission is very modest. The transmission when measured right after preparation is somewhat lower and slowly increases with time reaching the above stated values after 1-3 days. There are no changes in transmission with time, and the fibers can be stored in any position, straight or bent, without loss of transmission.

It is also possible to block the end of the tube (which is not coupled to the laser) with a transparent (to infrared and visible light) plastic film (e.g., polyethylene) which may be soldered to the tube, or with a ZnSe window glued to the tube.

Further improvements of hollow waveguides are described, for example, in U.S. Pat. No. 5,497,440 to Croitoru et al. Reference is now made to FIGS. 6-10, which are illustrations of the hollow waveguide described in the above-referenced patent.

The laser beam delivery system illustrated in FIG. 6 comprises a laser source 58, an adaptor assembly 60, a hollow laser beam waveguide 62 coupled at one end by adaptor assembly 60 to laser source 58, and a handpiece 64 carried at the opposite end of hollow laser beam waveguide 62. Laser source 58 may be a source of infrared radiation, such as produced by a CO₂ or Er-YAG laser (or a He—Ne aiming laser), as now being extensively used in surgical operations, as well as in material processing (e.g., cutting and welding equipment).

Adaptor assembly 60 is more particularly illustrated in FIG. 7. It includes, at one end, a fitting 66 for coupling to laser source 58, and at the opposite end, a rigid tube 68 coupled to the proximal end of hollow laser beam waveguide 62. Waveguide 62 may also be inserted into rigid tube 68. Adaptor assembly 60 further includes a gas fitting 70 between coupling fitting 66 and rigid tube 68. Coupling fitting 66 is formed with a gas inlet 69 for inletting a cooling gas, which gas is directed through hollow laser beam waveguide 62 onto the working area at the distal end of the waveguide.

Rigid tube 68 is at least 10 cm in length. It is intended to prevent producing a sharp bend at the proximal end of hollow laser beam waveguide 62 attached to the adaptor assembly or inserted into it. Preferably, rigid coupling tube 68 is from 10 to 20 cm in length.

Coupling fitting 66 preferably includes a lens 72 for focusing the laser beam from laser beam source 58 into the proximal end of hollow laser beam waveguide 62.

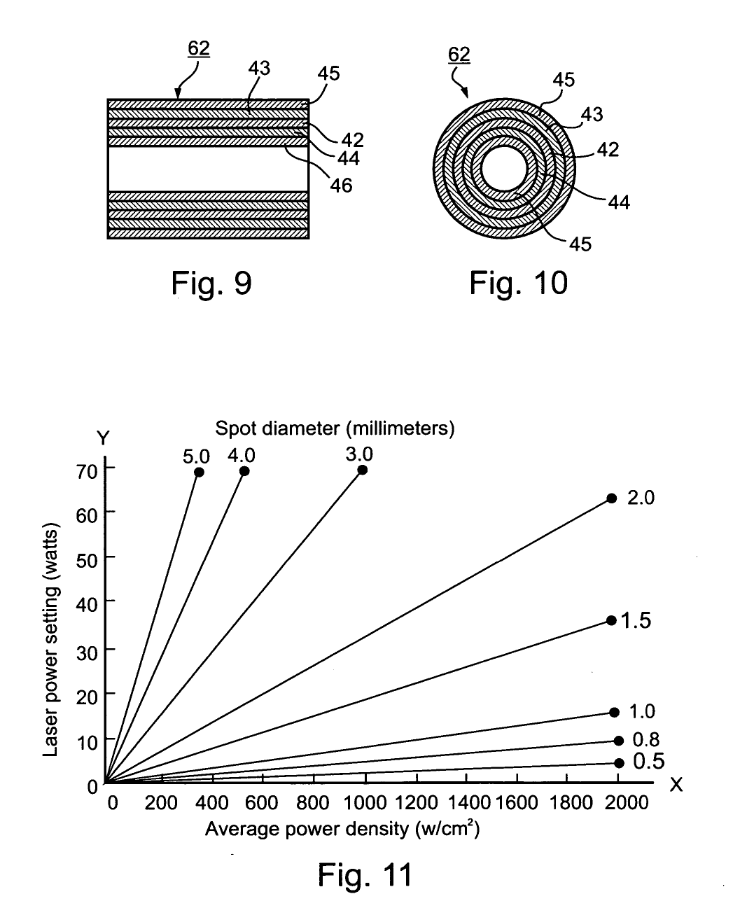

The structure of hollow laser beam waveguide 62 is more particularly illustrated in FIGS. 9 and 10. It includes a flexible, hollow plastic tube 42; a thin metal layer 44 on the inner surface of hollow plastic tube 42; and a flexible insulating layer 43 on the outer surface of hollow plastic tube 42. Preferably, the flexible, hollow plastic tube 42 is comprised of a fluoroethylene polymer, such as polytetrafluorethylene, but it could be, for example, one of the other materials mentioned in above-cited U.S. Pat. No. 4,930,863. A preferred material is “Teflon” (Reg. ™ of Dupont). An alternative preferred material is fused silica.

Flexible layer 43 over hollow plastic tube 42 should be of a material capable of absorbing infrared radiation and should prevent kinking of hollow plastic tube 42. A preferred material for this layer is silicone rubber.

Hollow laser beam waveguide 62 further includes a flexible metal sheath 45 over the outer surface of the flexible insulating layer 43. Metal sheath 45 has a darkened outer surface, e.g., black paint, to prevent reflection of stray radiation. This metal sheath may be, for example, a flexible metal braid, or a flexible metal spiral.

The thin metal layer 44 on the inner surface of hollow plastic tube 42 is preferably of silver, as described in prior U.S. Pat. No. 4,930,863. In that case, thin dielectric film 46 on the inner surface of metal layer 44 is preferably a silver halide, such as silver iodide, and is preferably applied according to the chemical processes described in that patent.

The handpiece 64 carried at the distal end of hollow laser beam waveguide 62 is grasped by the surgeon, or other user of the laser beam delivery system, for directing the laser beam to the desired working area. As shown in FIG. 8, it includes a graspable handle 74, and a deformable extension 76 capable of being manually reshaped (i.e., bent) to support the outlet end of the handpiece in the desired working area.

Other suitable hollow waveguides or IR fibers may include, for example, those described by Harrington et al. and by Gannot and Ben-David in the following references: Harrington et al., “Transmission properties of hollow glass waveguides for the delivery of CO₂ surgical laser power,” IEEE Journal of Selected Topics in Quantum Electronics, 1999 5(4): 948-953; Nubling and Harrington, “Hollow-waveguide delivery systems for high-power, industrial CO₂ lasers,” Applied Optics, 1996 35(3): 372-380; Harrington, A review of IR transmitting, hollow waveguides, Fiber and Integrated Optics, 2000 19(3): 21 1227, Gannot and Ben-David, “Optical fibers and wavelguides for medical applications,” Biomedical Photonics Handbook, chapter 7, pp 7-0.1:7-22 CRC press, 2003 and IR fibers and their applications by James A. Harrington (to be released in 2004).

It should be readily apparent that the above descriptions are for exemplary purposes only, and that any suitable flexible conduit for near or mid-IR laser delivery fall within the scope of the present invention.

Surgical Components

Several devices within system 100 are typical surgical instruments used for minimally invasive surgery. These include laser generator 140, gas insufflator 180 and surgical suction provider 210.

A. Laser Generator

Laser generator 140 and optic coupler 150 are used for providing hollow waveguide 130 with a laser beam for therapeutic applications to target tissue 110.

The ability of a surgical laser to incise, coagulate, or vaporize tissue depends on the concentration of power in the laser beam. Power density (expressed in W/cm²) describes the amount of laser power that is distributed over the cross-sectional area of the laser beam on the tissue being treated.

In general, during surgical applications, power densities below 500 mW/cm² tend to be used for tissue coagulation, whereas power densities of 1 kW/cm² and above are needed for vaporization and incision of tissues.

Reference is now made to FIG. 11, which is a graphical illustration of the relationship between laser power, beam diameter, and average power density. Average power density is depicted on the X-axis and laser power setting (in Watts) is depicted on the Y-axis. Values are shown for varying diameters of the beam. As is shown in the graph, power density increases by a factor of 4 when the beam diameter is halved. Varying spot size may be accomplished by changing the distance between the tissue being treated and the focusing lens of the optic fiber.

Furthermore, the duration of exposure is crucial, as the total amount of energy is directly related to the time, as formulated below:
Energy (Joules)=power (W)×time(s)

When using extremely high-power densities to obtain tissue ablation, the depth of vaporization is linearly correlated to exposure time.

Photothermal interactions are due to conversion of the laser energy into heat, and diffusion of heat within the tissue. When laser penetration depth is less than the laser spot radius, the thermal relaxation (also called diffusion) time tₚ can be defined as:

$${t}_{p} = \frac{{Z}^{2}}{4 {α}_{1}}$$

where Z is the thermal penetration depth of laser light in tissue and α₁ is the thermal diffusivity.

For laser pulses shorter than the thermal relaxation time, the distribution of thermal energy is determined by the light distribution. If the pulse duration is longer than the thermal relaxation time, the thermal energy propagates into tissue during the laser pulse. The thermal relaxation time is a constructed parameter, which emerges from the tissue parameters, and varies as the tissue varies at a constant laser light. A high repetition rate for the laser pulses can evoke an additional increase in temperature if the rate of heat transport is less than the rate of heat generation.

The thermal relaxation time, for instance, in water, with an optical penetration depth D=0.013 mm (shorter than the spot diameter) and a thermal diffusivity α₁=0.0015 cm²/sec, for CO₂ laser, is:

$${t}_{p} = \frac{{D}^{2}}{4 {α}_{1}} = \frac{{0.0013}^{2}}{4 * 0.0015} = 281.6 μ sec$$

Thus, the spot diameter and the laser pulse duration are factors in the photothermal effects on tissue.

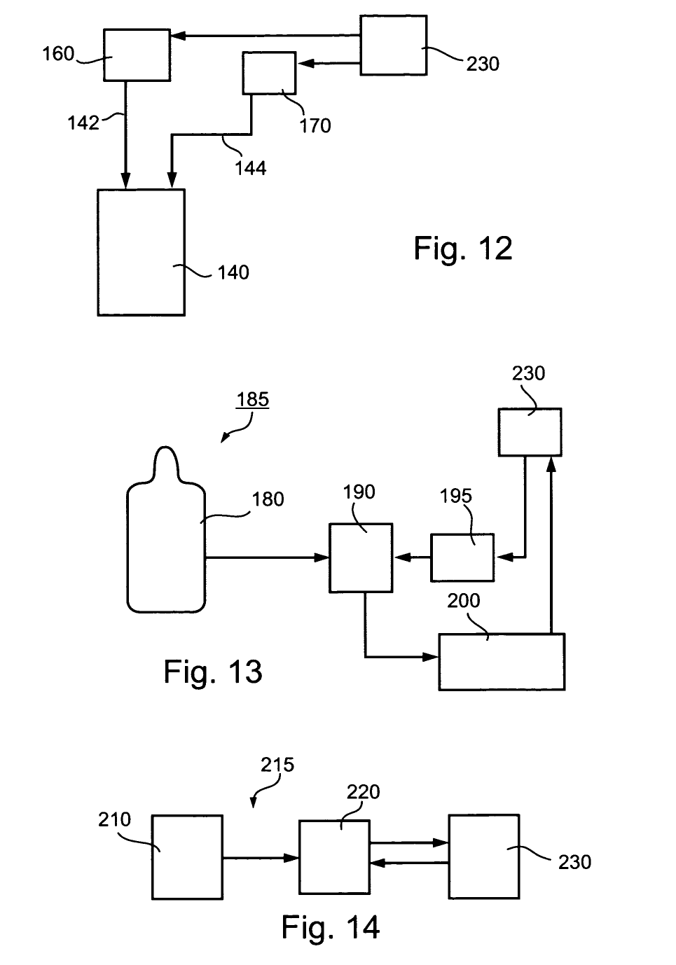

Reference is now made to FIG. 12, which is a block diagram illustration of an adjustable laser system in accordance with an embodiment of the present invention. A laser generator 140, such as the Sharplan 1041S CO₂ Laser Generator 0-100 w (Lumenis, Yokneam, Israel), is connected to a programmable power supply 160, such as the Goodwill PPT-3615 Programmable Power Supply (Good Will Instrument Co., Ltd. (GW), Taiwan) and is further connected to a function generator 170, such as the Stanford Research System DS335 Synthesized Functions Generator (Stanford Research Systems, Inc., Sunnyvale, Calif. USA). In a preferred embodiment, the laser user interface is bypassed. Laser generator 140 is activated and controlled by two input channels: an analog channel 142 and a digital channel 144, each of which is wired directly to laser electronics. Analog channel 142 controls the laser pulse amplitude, while digital channel 144 controls the laser pulse rate and duty cycle. Power supply 160 is in communication with laser generator 140 via analog channel 142, and function generator 144 is in communication with laser generator 140 via digital channel 144. Both power supply 160 and function generator 170 are controlled and monitored by controller 230. Any other combination of manufacturers of parallel equipment is possible, and would fall within the scope of the invention.

In an alternative embodiment, laser parameters are controlled entirely by software or hardware, bypassing the need for power supply 160 and function generator 170, as follows. Labview sub VI can directly control pulse rate and duty cycle through NI-DAQ and an external connection board, wired to digital channel 144; and Labview sub VI can directly control pulse amplitude and on/off through NI-DAQ and an external connection board, wired to analog channel 142.

The laser beam generated via laser generator 140 is sent through an optic coupler, such as, for example, made by Lumenis (Yokneam, Israel) and into hollow waveguide 130 for administration through endoscope 120 to target tissue 110.

B. Gas Insufflator

Reference is now made to FIG. 13, which is a block diagram illustration of an insufflating system 185 in accordance with an embodiment of the present invention. Insufflating system 185 functions to insufflate the sphere in which the surgery is performed, particularly with CO₂ gas. It additionally enables the use of gas for cooling. Insufflating system 185 includes a CO₂ gas insufflator 180 connected to a proportional valve 190. Proportional valve 190 sends gas flow through pressure and flow meters 200 through endoscope 120, and into target tissue 110. Pressure and flow meters 200 include a low pressure regulator, a pressure gauge, and an electrical flow meter. Proportional valve 190 is regulated by a valve controller 195, which is controlled by controller 230. Further, pressure and flow meters 200 send feedback to controller 230 to aid in regulation of insufflating system 185.

Insufflating the cavity, generally by CO₂ purge gas, allows the management of the endoscope tip inside the cavity. By circulating the atmosphere, insufflation averages heat dispersion within the cavity.

CO₂ gas minimizes the risk of embolism but also partially absorbs CO₂ laser energy. The phenomenon, known as resonant absorption, limits the power available for tissue ablation. With a laser power of 30 Watts, for example, the loss of power is considerable and it grows as power climbs.

Cooling the CO₂ laser waveguide is obtained by injecting flow (air, N₂ or water) into the cavity, through the fiber's hollow core. In terms of heat dispersion, this flow has principally a similar effect to that of insulating gas, but on a lower scale.

C. Surgical Suction System

Reference is now made to FIG. 14, which is a block diagram illustration of a surgical suction system 215, in accordance with an embodiment of the present invention. Surgical suction system 215 includes a surgical suction provider 210, as found in a typical operating room, such as, for example, Acu-E-Vac II Surgical Suction (Acuderm, Inc., Fort Lauderdale, Fla., USA), a proportional vacuum valve and electrical vacuum meter 220 connected to surgical suction provider 210, and a controller 230 for control and monitoring of suction provided through endoscope 120 to target tissue 110. The suction evacuates smoke, vapor and tissue particles, to unmask the treated area and to reduce the heat convection within the cavity.

III. Measurement of Photothermal Effects

Thermography Basics

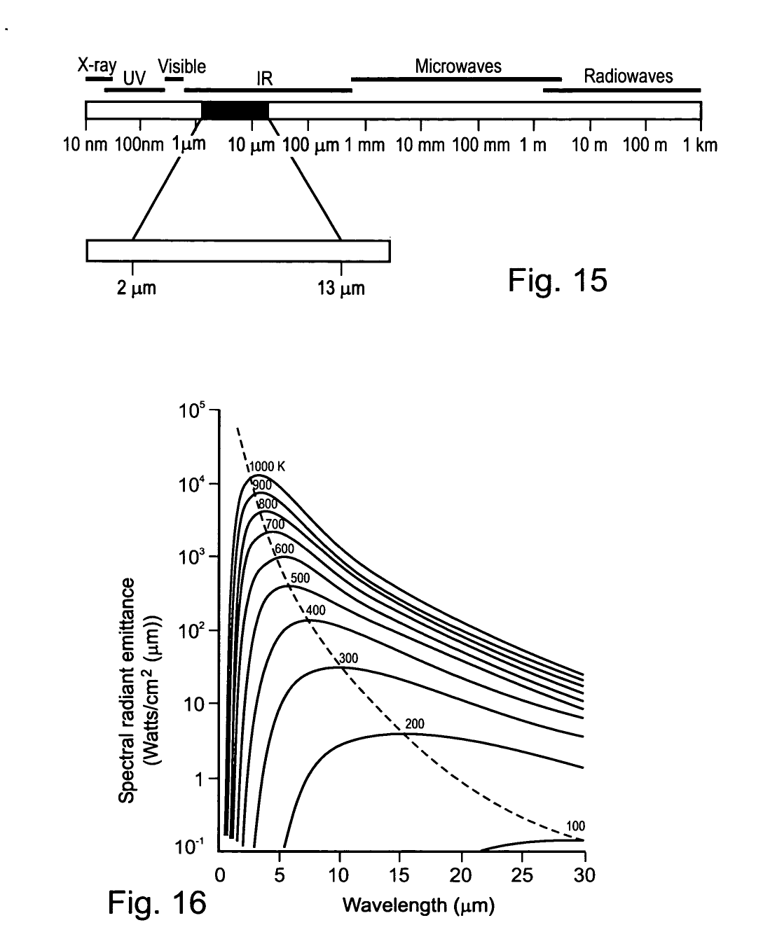

Thermal imaging utilizes an array of infrared detectors to detect radiant emittance. Reference is now made to FIG. 15, which is an illustration of the electromagnetic spectrum, showing the range of detection for thermal imaging as between 2 μm and 13 μm.

Radiation emitted from a blackbody is expressed through Planck's law, Wien's displacement law, and the Stefan-Boltzmann law.

Planck's Law

Max Planck (1858-1947) was able to describe the spectral distribution of the radiation from a blackbody by means of the following formula:
W_{λb}=2πhc² 10⁻⁶/λ⁵(e^{hc/λkT}−1) (Watts/m² μm)
where:
W_{λb}=the blackbody spectral radiant emittance at wavelength λ.
c=the velocity of light=3×10⁸ n/sec.
h=Planck's constant=6.6×10⁻³⁴ Joule sec.
k=Boltzmann's constant=1.4×10⁻²³ Joule/K.
T=the absolute temperature (K) of a blackbody.
λ=wavelength (m).

Planck's formula, when plotted graphically for various temperatures, produces a family of curves, as shown in FIG. 16. Following any particular Planck curve, the spectral emittance is zero at λ=0, then increases rapidly to a maximum at a wavelength λₘₐₓ and decreases exponentially back to zero. The higher the temperature, the shorter the wavelength at which maximum occurs.

Wien's Displacement Law

Differentiating Planck's formula with respect to λ, and finding the maximum, yields:
λₘₐₓ=2898/T(μm)
This is Wien's formula, which expresses mathematically the common observation that colors vary from red to orange or yellow as the temperature of a thermal radiator increases. The wavelength of the color is the same as the wavelength calculated for λₘₐₓ. A good approximation of the value of λₘₐₓ for a given blackbody temperature is obtained by applying the rule-of-thumb 3000/T μm. For instance, the sun (˜6000 K) emits yellow light, peaking at ˜0.5 μm in the mid visible spectrum, while at room temperature (300 K) the peak of radiant emittance-lies at 9.7 μm, in the mid IR.

The Stefan-Boltzmann Law

Integrating Planck's formula from λ=0 to λ=∞, obtains the total radiant emittance (W_{b}) of a blackbody:
W_{b}=σT⁴ (Watts/m²)
where:
σ=the Stefan-Boltzmann constant=5.7×10⁻⁸ Watt/m²

This is the Stefan-Boltzmann formula, which states that the total emissive power of a blackbody is proportional to the fourth power of its absolute temperature. This means that even slight temperature differences between adjacent areas of a blackbody will result in large differences in radiant emittance.

Graphically, W_{b} represents the area below the Planck curve for a certain temperature. It can be shown that the radiant emittance in the interval λ=0 to λₘₐₓ is only 25% of the total.

Using the Stefan-Boltzmann formula to calculate the power radiated by the human body, at a temperature of 300 K and an external surface area of approximately 2 m², the power equals 1 kW. This power loss could not be sustained if it were not for the compensating absorption of radiation from surrounding surfaces, at room temperatures, which do not vary too drastically from the temperature of the body.

Real objects almost never comply with these laws over an extended wavelength region—although they may approach the blackbody behavior in certain spectral intervals. For example, white paint appears perfectly ‘white’ in the visible light spectrum, but becomes distinctly ‘gray’ at about 2 μm, and beyond 3 μm it is almost ‘black’.

Three processes can prevent a real object from acting like a blackbody: a fraction of the incident radiation α may be absorbed, a fraction ρ may be reflected, and a fraction τ may be transmitted. Since all of these factors are more or less wavelength dependent, the subscript λ is used to imply the spectral dependence of their definitions.

Thus: (i) The spectral absorbance α_{λ}=the ratio of the spectral radiant power absorbed by an object to that incident upon it; (ii) The spectral reflectance ρ_{λ}=the ratio of the spectral radiant power reflected by an object to that incident upon it; and (iii) The spectral transmittance τ_{λ}=the ratio of the spectral radiant power transmitted through an object to that incident upon it

The sum of these three factors must always add up to the whole at any wavelength, so we have the relation:
α_{λ}+ρ_{λ}+τ_{λ}=1

For opaque materials τ_{λ}=0, and the relation simplifies to:
α_{λ}+ρ_{λ}=1

Another factor, called the emissivity, is required to describe the fraction ε of the radiant emittance of a blackbody produced by an object at a specific temperature. Thus, we have the following definition: The spectral emissivity ε_{λ}=the ratio of the spectral radiant power from an object to that from a blackbody at the same temperature and wavelength. Expressed mathematically, this can be written as the ratio of the spectral emittance of the object to that of a blackbody as follows:
ε_{λ}=W_{λ0}/W_{λb}

Generally, there are three types of radiation source, distinguished by the ways in which the spectral emittance of each varies with wavelength, as follows:
(i) A blackbody, for which ε₁=ε=1.
(ii) A graybody, for which ε₁=ε=constant less than 1.
(iii) A selective radiator, for which ε varies with wavelength.

According to Kirchhoff's Law, for any material the spectral emissivity and spectral absorption of a body are equal to any specified temperature and wavelength. That is:
ε_{λ}=α_{λ}

From this, for an opaque material (since α_{λ}+ρ_{λ}=1):
ε_{λ}+ρ_{λ}=1

For highly polished materials, approaches zero, so that for a perfectly reflecting material (i.e. a perfect mirror):
ρ_{λ}=1

For a graybody radiator, the Stefan-Boltzmann formula becomes
W=εσT⁴ (Watt/m²)

This states that the total emissive power of a graybody is the same as a blackbody at the same temperature reduced in proportion to the value of ε from the graybody.

For a non-metallic, semi-transparent body, in the form of a thick flat plate of plastic material, the calculations are as follows. When the plate is heated, radiation generated within its volume must work its way toward the surfaces through the material in which it is partially absorbed. Moreover, when it arrives at the surface, some of it is reflected back into the interior. The back-reflected radiation is again partially absorbed, but some of it arrives at the other surface, through which most of it escapes; part of it is reflected back again. Although the progressive reflections become weaker and weaker they must all be added up when the total emittance of the plate is sought. When the resulting geometrical series is summed, the effective emissivity of a semi-transparent plate is obtained as
ε_{λ}=(1−ρ_{λ})(1−τ_{λ})/(1−ρ_{λ}τ_{λ})

When the plate becomes opaque (τ_{λ}=0) this formula is reduced to the single formula:
ε_{λ}=1−ρ_{λ}

This last relation is a particularly convenient one, because it is often easier to measure reflectance than to measure emissivity directly.

Fiber Bundles

Coherent fiber-optic bundles, composed of oxide glass fibers, have been fabricated for many years, using mandrel wrap ping and leached bundle technologies. These bundles have been used to transmit high resolution images for a variety of applications. There has, however, been very little work to extend the wavelength range of imaging fiber bundles to wavelengths greater than 2.5 μm. Most of the current infrared imaging fiber bundles (IRIFB) for wavelengths incorporating 9-10 μm are fabricated from chalcogenide glass fibers. Nishi et al. “Coherent Infrared Fiber Image Bundle,” Applied Physics Letters, 1991, 59(21): p. 2639-2641, and Hilton, A.R. “Infrared Imaging Bundles with Good Image Resolution,” in SPIE—Optical Fiber and Sensors for Medical Applications, 2001, have made coherent IRIFBs comprising several thousand As₂S₃ fibers. Katzir and Rave have extruded polycrystalline silver halide fibers into coherent core-clad bundles, as described in: Rave and Katzir, “Ordered bundles of infrared transmitting silver halide fibers: attenuation, resolution and crosstalk in long and flexible bundles,” Optical Engineering, 2002, 41(7): p. 1467-1468; and Rave et al., “Thermal imaging through ordered bundles of infrared transmitting silver-halide fibers,” Applied Physics Letters, 2000, 76(14): p. 1795-1797.

Most recently Gopal et al. “Coherent IR bundles fabricated from hollow glass waveguides (HGWs),” Optical Engineering, Accepted November 2003, have employed Ag/AgI coated hollow glass waveguide (HGW) technology to form coherent IRIFB. An initial ex-vivo study with HGW IRIFB (900 bundled fibers of 65 μm ID each) has demonstrated its ability to transfer high resolution IR images of a laser-based tissue ablation.

Based on Wein's law (λₘₐₓ=2898/T) and in-vivo studies the spectral response should peak between 9.1 μm (45° C.) and 9.4 μm (35° C.) for diagnostic purposes. Optical transmission should also cover the range of 7.8 μm (100° C.—at which tissue evaporation starts) to 9 μm (46° C.—at which thermal side effects may start), applying to temperature distribution along the irradiated tissue zone and the surroundings during thermally-based ablation. Consequently, the IRIFBs, as well as all setup components, should have the spectral range of 7.5-9.4 μm. The spatial resolution requirements may vary based on the tissue target. For example, in order to detect the presence of malignancies in the order of 1 mm, IRIFBs (and the setup as a whole) of better resolution than 3 lines/mm (333 μm) should reasonably satisfy. Clearly, fine thermal resolution is crucial for certain applications. This parameter, expressed in terms of Minimal Resolvable (and Detectable) Temperature Differences (MRTD and MDTD), are evaluated by adequate testing equipment of 0.001° C. resolution.

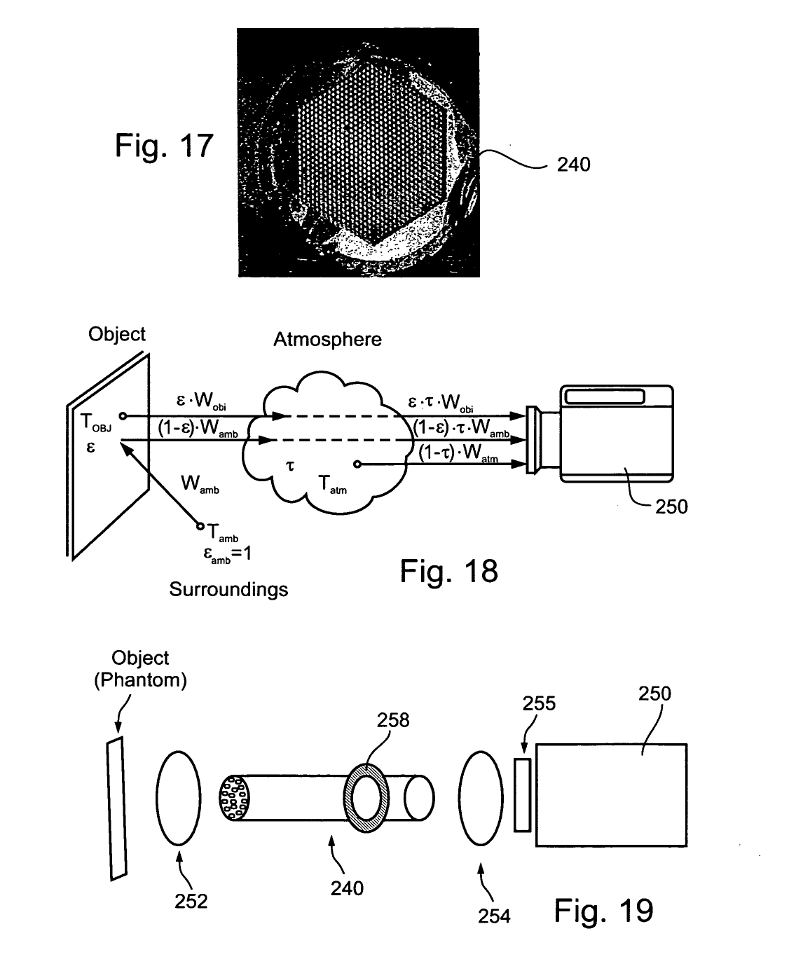

Reference is now made to FIG. 17, which is a cross-sectional illustration of an IRIFB 240 in accordance with a preferred embodiment of the present invention. The IRIFB shown includes 900 hollow fibers, each of a diameter of 65 um, although other diameters are possible. Various mid-IR IRIFBs are available, including Ag/AgI coated HGW IRIFBs, incorporating 30 to 900 elements of 300-40 μm ID each (respectively), as well as polycrystalline silver halide core-clad IRIFBs of 100-900 elements (core fibers), 251-100 μm ID each and flexible IRIFBs of 36-100 elements, 70-40 μm ID each.

Details regarding fabrication of hollow IRIFBs as described in Gopal et al., are included herein. Briefly, rigid arrays composed of ordered glass capillary tubes can be purchased from, for example, Collimated Holes, Inc. (Campbell, Calif., USA). In one example, an array having 500 holes with an individual bore size of 150 μm and another array having 900 holes with a bore size of 50 μm are used. The length of the as received tubing may be as long as 1 m, but in a preferred embodiment may be coated for a length of 2 cm to 20 cm.

The active core area of the bundles determines the actual imaging area. The calculated core areas for the two capillary arrays that we used are given in Table 1

TABLE 1

| Collimated | | | | |
|---|---|---|---|---|
| Hole bundle | Bore, μm | OD bundle, mm | No. of fibers | % core area |
| CH50900 | 50 | 3 | 900 | 25 |
| CH150500 | 150 | 6 | 500 | 31.2 |

Normally for good resolution a bore size nearer 25 μm is desirable. Additionally, it is generally desirable to have an active core region greater than 50%. This would involve fabricating bundles made from tubing with a smaller capillary wall thickness. In the bore size tubing described above, the wall thickness should be between 5 and 10 μm to achieve an active core area of at least 50%.

The same liquid phase chemistry procedure used to make single bore HGWs is used to coat the rigid coherent bundles. The first thin film deposited is silver followed by a conversion of some of the silver film into AgI for enhanced reflectivity. The thickness of the AgI film is tailored to give low loss across a broad spectral range. This is generally desirable for most IR imaging applications. The AgI film produces interference peaks, from which the film's thickness can be estimated. The bundles described hereinabove have been shown to transmit in the long 8 to 12 μm wavelength region.

Details regarding fabrication of core-clad IRIFBs, as described in Rave et al., “Infrared photonic crystal fiber,” Applied Physics Letters, 83(10): 2003 are as follows. Briefly, single crystals of AgClₓBr₁₋ₓ are extruded through small dies to form unclad fibers. These polycrystalline fibers are flexible, non-toxic, and non-hygroscopic, and their transmission losses are of the order of α=0.2 dB/m at λ=10.6 μm. The design guidelines for a polycrystalline fiber in the mid-IR range are as follows:
a) In order to keep its flexibility, the diameter of the PCF should be less than 1 mm.
b) The effective area of the core should be relatively large.
c) The number of fiber optic elements in circles around the core should be relatively large, to operate as an effective cladding layer.

An exemplary embodiment of a core-clad IRIFB is designed as follows. The material used is AgBr (n₁=2.16). Instead of the traditional air holes in the clad, AgCl (n₂=1.98) fibers with a diameter of 50 mm is incorporated. The AgCl fibers are arranged along two circles, with a total of 30 elements, and a pitch of 110 mm. The polycrystalline fiber can be fabricated in four steps:
1) Extrusion of several meters of C/C fiber, of outer diameter 1 mm, using the “rod in tube” method. In this fiber the refractive index of the core AgCl is smaller than that of the AgBr cladding.
2) Cutting of the C/C fiber into 5 cm segments.
3) Extrusion of an unclad AgBr fiber and cutting of the fiber into 5 cm segments.
4) Organization of all the small segments together in the following order: six segments from the unclad AgBr fiber arranged at the center and 30 segments from the C/C fiber arranged around the center, in two concentric circles. The whole arrangement of segments is used as a new preform, extruded through a small die to form the silver halide PCF. Using this method, PCFs are obtained of diameter 1 mm and lengths of the order of 1 m.

The fabrication of IRIFBs is not limited to the described methods, but rather, any suitable method for fabrication of IRIFBs is contemplated, and would fall within the scope of the present invention.

IR Camera

When viewing an object, a camera receives radiation from three sources: the object itself, the surroundings, reflected by the object surface, and the atmosphere. The atmosphere along the viewing medium attenuates contributions of the first two. Reference is now made to FIG. 18, which is a schematic illustration of a camera 250 and thermographic measurement. This description of the measurement situation neglects other emitters such as sun light scattering in the atmosphere or stray radiation from intense radiation sources outside the field of view. These disturbances are difficult to quantify, however, and in most cases they are small enough to be neglected. In cases where they are not, the measurement configuration is likely to be such that the risk for disturbance is obvious. Changing the viewing direction or shielding off intense radiation sources will avoid them. Using this model, we can derive a formula for the calculation of the object temperature from the calibrated camera output.

Assuming the received radiation power W from a blackbody source of temperature T_{source} on short distance generates a camera output signal U_{source} that is proportional to the power input (power linear camera), we can write:
U_{source}=C·W(T_{source})
or, with simplified notation:
U_{source}=C·W_{source}
where C is a constant.

Should the source be a graybody with emittance ε, the received radiation would consequently ε·W_{source}

The three collected radiation power terms:
(1) Emission from the object=ε·τ·W_{obj}
   where ε is the emittance of the object and τ is the transmittance of the atmosphere. The object temperature is T_{obj}.
(2) Reflected emission from ambient sources=(1−ε)·τ·W_{amb}
   where (1−ε) is the reflectance of the object. The ambient sources have the temperature T_{amb}.

It has been assumed that the temperature T_{amb} is the same for all emitting surfaces within the hemisphere seen from a point on the object surface. This is sometimes a simplification of the true situation. It is, however, a necessary simplification in order to derive a workable formula, and T_{amb} can theoretically be to given a value that represents an efficient temperature of a complex surrounding.

It is additionally assumed that the emittance for the surroundings=1. This is correct in accordance with Kirchhoffs law: All radiation impinging on the surrounding surfaces will eventually be absorbed by the same surfaces. Thus the emittance=1.
(3) Emission from the atmosphere=(1−τ)·τ·Wₐₜₘ
   where (1−τ) is the emittance of the atmosphere. The temperature of the atmosphere is Tₐₜₘ

The total received radiation power can now be written:
Wₜₒₜ=ε·96 ·W_{obj}+(1−ε)·τ·W_{amb}+(1−τ)·Wₐₜₘ

Each term is multiplied by the constant C and the product C·W is replaced by the corresponding U according to the same equation:
Uₜₒₜ=ε·τ·U_{obj}+(1−ε)·τ·U_{amb}+(1−τ)·Uₐₜₘ
or:
U_{obj}=Uₜₒₜ/ε·τ+U_{amb}(ε−1)/ε+Uₐₜₘ(ε−1)/ε·τ
where:
U_{obj}—Calculated camera output voltage for a blackbody of temperature T_{obj} i.e. voltage that can be directly converted into requested object temperature.
Uₜₒₜ—Measured camera output voltage for the actual case
U_{amb}—Theoretical camera output voltage for a blackbody of temperature T_{amb} according to calibration
Uₐₜₘ—Theoretical camera output voltage for a blackbody of temperature Tₐₜₘ according to calibration

This is the measurement formula of a thermal camera 250 used in accordance with a preferred embodiment of the present invention.

IRIFB and Camera Setup

Reference is now made to FIG. 19, which is a schematic illustration of an IRIFB 240 and an infrared camera 250 and their relative setup.

The setup includes a camera 250 such as, for example, Thermacam SC500 IR camera (FLIR Systems AB, Danderyd, Sweden), IR ZnSe imaging and magnifying lenses 252, 254, IRIFB 240, iris diaphragm 258 and a tissue target 110. The IR camera (7.5-13 μm, 0.07° C.@30° C., 14 bits, 4 images/sec) is equipped with a close-up lens 255 (130 μm resolution in a preferred embodiment) and an optional cutoff filter above 9.9 μm to avoid laser reflections at 10.6 μm. The entire setup is controlled by customized applications (FLIR's Thermacam Researcher and Labview-based program) for recording and analyzing the IR images. The close-up lens 255 magnifies the image (up to 18 μm resolution) received via the IRIFB and a second, optional lens (not shown) focuses the object's image at the distal tip of the IRIFB. The optical system is preferably covered by a non-reflective case.

The entire system is incorporated as part of the feedback mechanism of system 100, providing both visual and quantified data for monitoring and control of laser parameters, suction volume and insufflation flow rate, each of which can be controlled either manually or automatically by software applications, in order to maintain the desired temperature in the interaction zone and to protect the surrounding tissue from thermal side effects.

Central Control System

Heat dispersion within human tissues during laser radiation is a multi-parameter problem, and depends on several broad categories, including the energy transmission system, the tissue characteristics, and the interface between the transmission system and the tissue characteristics. More specifically, the following parameters must be taken into account:

For the energy transmission system, the key parameters include the laser power, the mode (continuous versus pulse), the duration of laser transmission when in continuous mode, the pulse duration, shape and frequency and duty cycle when in the pulse mode, the fiber characteristics (straight versus angulated, diameter and profile, perfusion and attenuation), and the hit angle.

For the tissue characteristics, the key parameters include the composition (mainly the water percentage of every layer induced), the crater deepness and shape, and the initial temperature

For the interface between the transmission system and the tissue characteristics, the key parameters include the cavity volume and geometry of surrounding tissue, the distance between the fiber edge and the irradiated tissue, the gas content, the atmospheric pressure (or vacuum), the gas flow rate and mode.

Any parameters that are controllable or variable can theoretically be varied via the control system of the present invention. However, since in practice the surgeon controls the laser power, the transmission duration, the pulse mode, the CO₂ insufflating and the suction volume and in a limited way controls the angle and the distance between endoscope and tissue, those parameters are focused on for the purposes of the present invention.

The following parameters are adjustable with respect to the laser directed at the target tissue. First, the laser power can be controlled by power supply 160 or directly by controller 230 by adjusting the voltage. In a preferred embodiment, the voltage ranges from 0-5 volts. The transmission mode (continuous wave versus pulse wave) is controlled by function generator 170 or by a pulse generator circuit. The duration of the wave (in continuous mode) and the pulse rate and duty cycle (in pulse mode) are also controlled either by function generator 170 or by a pulse generator circuit. In pulse mode, pulse duration may vary between several μsec to several seconds. The beam diameter is usually in a range of 1-8 mm, and at least partially depends on the fiber diameter, which may vary between 50-2000 μm. The hit angle is manually controllable by the physician performing the procedure. The hit angle may be perpendicular, or it may be within a range of 45-70 degrees.

Other parameters which are adjustable include the gas insufflation, typically 0-10 lpm and the suction volume, which can vary between 0-300 mmHg, both of which are controllable by controller 230.

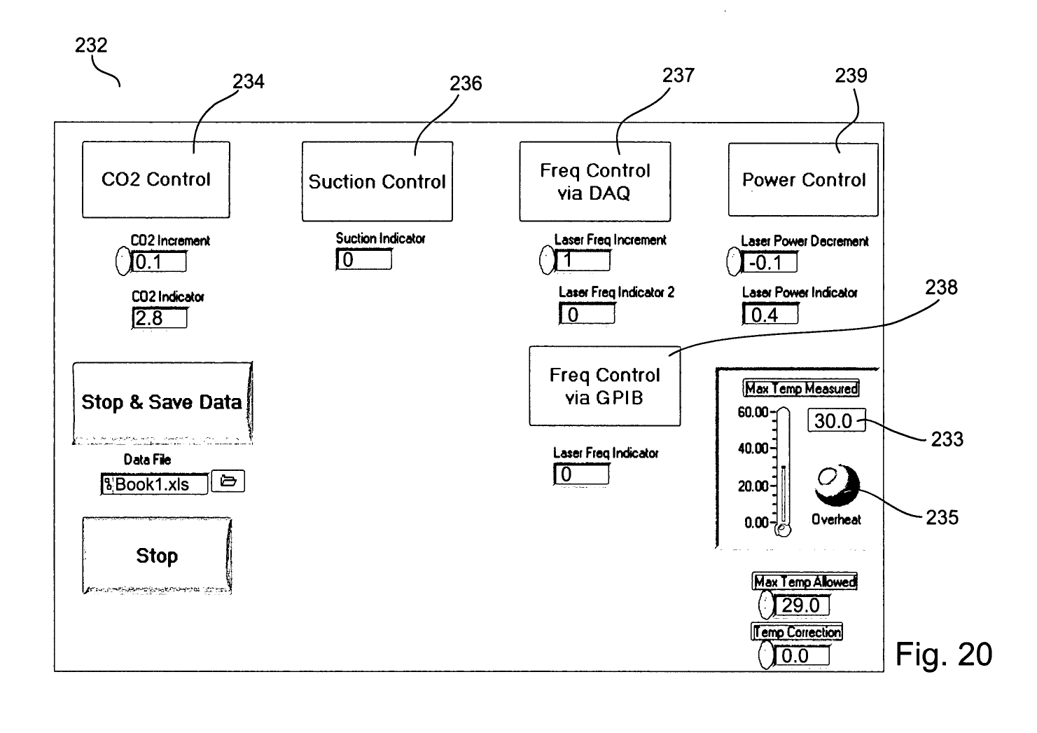

All of the adjustable parameters of system 100 are controlled by a central control system, which includes a processor 235. Processor 235 may include software, hardware, or any combination thereof. Furthermore, processor 235 can include a controller 230 and a thermographic analyzer 270. Each of these components can be comprised of software, hardware, or a combination of both. In a preferred embodiment, controller 230 is comprised of, for example, a Labview program used to control the various parameters. Reference is now made to FIG. 20, which is an illustration of a Labview main panel 232 for control, data acquisition and optimization.

As shown in FIG. 20, the main panel includes a CO₂ control 234, a suction control 236, frequency control 237 using DAQ, frequency control using 238 GPIB, and power control 239. Additionally, the main panel includes a gauge 233 for temperature measurement and an indicator 235 for overheating.

The system 100 further includes a thermographic analyzer 270, which is suitable for reading measurements from IR camera 250, and incorporating the data into a usable format. In one embodiment, thermographic analyzer 270 is a component of processor 235. In another embodiment, thermographic analyzer 270 is a separate configuration, comprised of software, hardware, or a combination. For example, thermographic analyzer 270 may be a ThermaCAM Researcher Application software program. For example, data may be mapped, giving a two- or three-dimensional color-coded representation of thermal properties-during the procedure. Alternatively, ail data can be sent to controller 230 for further adjustment of the adjustable parameters.

Additional objects, advantages, and novel features of the present invention will become apparent to one ordinarily skilled in the art upon examination of the following examples, which are not intended to be limiting. Additionally, the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

The relative effects of every controllable component during an MIS on the thermal properties and maximal temperatures of surrounding tissue were examined by simulating the in-vivo environment in vitro.

Tissue Phantoms

Tissue phantoms were used to simulate tissue thermal properties. Specifically, polyacrylamide gel, agar gel, and in-vitro biological specimens taken from porcine and cow organs were used.

Phantoms are usually homogeneous, and therefore do not normally possess the complex structures that exist throughout tissue. Therefore, tissue substitutes do not necessarily reflect the true complexity and hence response of tissue during laser therapy. However, they do provide a qualitative means of testing the parameters and assumptions made in mathematical models and may indicate generally important features of opto-thermal processes in tissue. Phantoms may also be used to demonstrate and elucidate the transient effects of interstitial laser heating. For instance, heating of tissue during interstitial laser photocoagulation produces a coagulated region surrounded by healthy tissue. This occurs in a dynamic fashion, which depends upon a variety of factors such as the temperature dependent changes in optical properties of the tissue and changes in blood perfusion. Phantoms may therefore be used to investigate these effects in a controlled fashion where the complexities of in vivo studies can be avoided.

Polyacrylamide (PAA) gels used as tissue phantoms give a more realistic simulation of tissue than to water. This gel has structural integrity, which can be varied by varying the water concentration (typically between 60 and 95%, i.e. 40 to 5% PAA). In addition the gel is optically transparent which allows visualization of the ablation process. Compared to other gels such as agar or gelatin, the biggest advantage of PAA is that it does not melt at elevated temperatures, which is important in ablation studies. However, acrylamide as a monomer is considered toxic, directly affecting the nervous system, and it may reasonably be considered to be a carcinogen. Acrylamide is readily absorbed through intact skin from aqueous solutions.

PAA phantoms have been used during near IR laser photocoagulation studies, evaluation of hyperthermia utilization, studies in laser interstitial thermal therapy for treating breast tumors, investigation of thermal events during CW argon laser ablation, and for other studies as well.

Agar/Agarose gels are generally used to simulate tissue optical properties. Yet it has served some thermal investigations, and is considered safe to work with. For instance, an Agar phantom possessing a cavity for simulating the stomach was employed to evaluate temperature distribution upon RF-heating. Such therapy was aimed at patients with advanced gastric cancer.

The gels are frequently combined with substances such as Intralipid and Naphthol green dye to imitate tissue properties. Intralipid is a weakly absorbing, highly scattering liquid emulsion that can be diluted with water to yield tissue-like scattering in the near IR. Naphthol green dye is an organic powder that can be dissolved in water and added to Intralipid suspension to yield absorption properties of tissues in the near IR.

Various phantoms were fabricated from combinations of plastic hemispheres and nylon constructions, layered with agarose gel or in-vitro porcine stomach samples. Agarose was fabricated as follows:

A quantity of 0.8 g agarose (Seakern HE agarose, FMC Bioproduct) and 36.6 ml PBS-phosphate buffered saline (J. T. Baker, cat. No. 5656) were heated and boiled while stirring to dissolve the agarose. The solution obtained was clear and free of bubbles. After cooling to 70° C., 3.4 ml Intralipid (Intralipid 20%, Pharmacia & Upjohn, cat. No. 406563A) was added to the solution while shaking the tube gently. The solution was then poured into petri plates.

Setup

An upper and a lower tissue sample were used. The samples had an initial temperature of approximately 24° C. Suction and CO₂ inlet tips were inserted through drilled holes. An IR camera, located beneath the phantom, was focused on the upper tissue sample through a nylon wrapped window, and recorded approximately four thermal images per second. The nylon wrap attenuated temperature readings by 2° C. (measured). The recorded files were then analyzed into graphs and tables. The trial was performed at various powers, with and without air injected through the fiber, in the presence or the absence of suction (40 mmHg) and at two different distances between tissues (2.8 cm, 4 mm).

In a first setup, using a fixed laser power, the air flow, suction and continuous/pulsatile waves were changed to explore each one's effect on heat dispersion.

In a second setup, only the laser power was altered in order to study its effect on thermal gradients versus time and location.

In a third setup, the time and power were varied, while other parameters remained constant.

Results

1^{st} Setup:

Assuming linear slopes during transmission periods of 7 seconds, average heating rate was calculated. ΔT was defined as the difference between initial temperature (when irradiation started) and the maximal temperature observed after 7 seconds. This was performed at a power of 15 watts. Results are summarized in Table 2.

TABLE 2
Average heating rate in the surrounding tissue at different
conditions; 15watts, 7 seconds elapsed

| 15 watts | | | | | | Pulsed |
|---|---|---|---|---|---|---|
| (750 w/ | | | | | | with |
| cm²), | Neither | | | | Pulsed, | Airflow |
| 7 sec | air nor | Airflow | Suction | Airflow & | 0.5 on/ | 0.5 on/ |
| elapsed | suction | 31 pm | 40 mmHg | suction | 0.5 off | 0.5 off |
| ΔT (° C.) | 7.5 | 5 | 2.5 | 1 | 2.2 | 1.8 |
| Av. slope | 1 | 0.7 | 0.35 | 0.14 | 0.3 | 0.25 |
| (° C./Sec) | | | | | | |

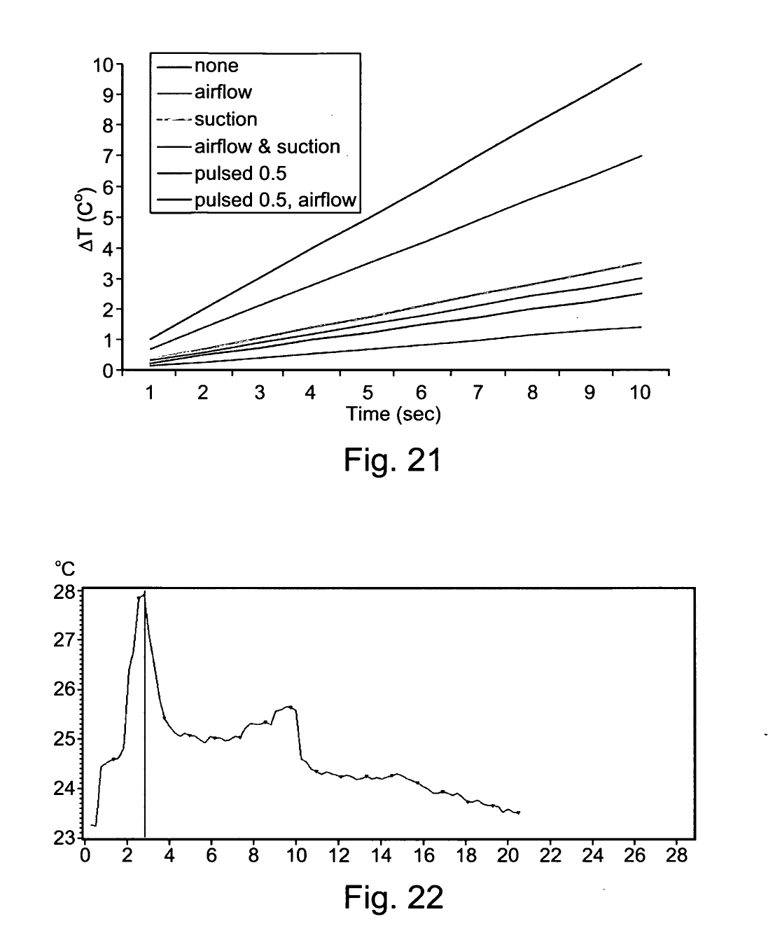

Reference is now made to FIG. 21, which is a graphical illustration of the effects of varying airflow, suction, and wave type on temperature changes. Lines were extrapolated via linear regression. As shown in FIG. 21, 31 pm airflow reduced the thermal gradient by 33%, 40 mmHg vacuum reduced it by 67% and both simultaneously by 80%. The suction influence will be described further hereinbelow.

The pulse mode of 0.5-dutycycle (half the energy) shrank the gradient by 64% with airflow injected and by 70% without it. Tissue thermal relaxation between pulses caused greater percentage than the expected 50.

Reference is now made to FIG. 22, which is a graphical illustration of the effect of suction on temperature changes. Suction was introduced at 2.5 seconds and at 6 seconds. It is clear from FIG. 22 that the maximum temperature drops dramatically when suction is activated, and rises again when vacuum becomes weakened. Suction efficiently reduces the temperature of the surroundings by eliminating steam.

2^{nd} Setup:

Results in temperature changes over 7 seconds for power of 15 watts, 6 watts and 4 watts are summarized in Table 3 below.

TABLE 3
Average heating rate in the surrounding tissue using
various powers; 7 seconds

| 7 sec | 15 watts | 6 watts | 4 watts |
|---|---|---|---|
| elapsed | (750 w/cm²) | (300 w/cm²) | (200 w/cm²) |
| ΔT (° C.) | 7.5 | 4.5 | 3 |
| Av. | 1 | 0.64 | 0.43 |
| slope | | | |
| (° C./Sec) | | | |

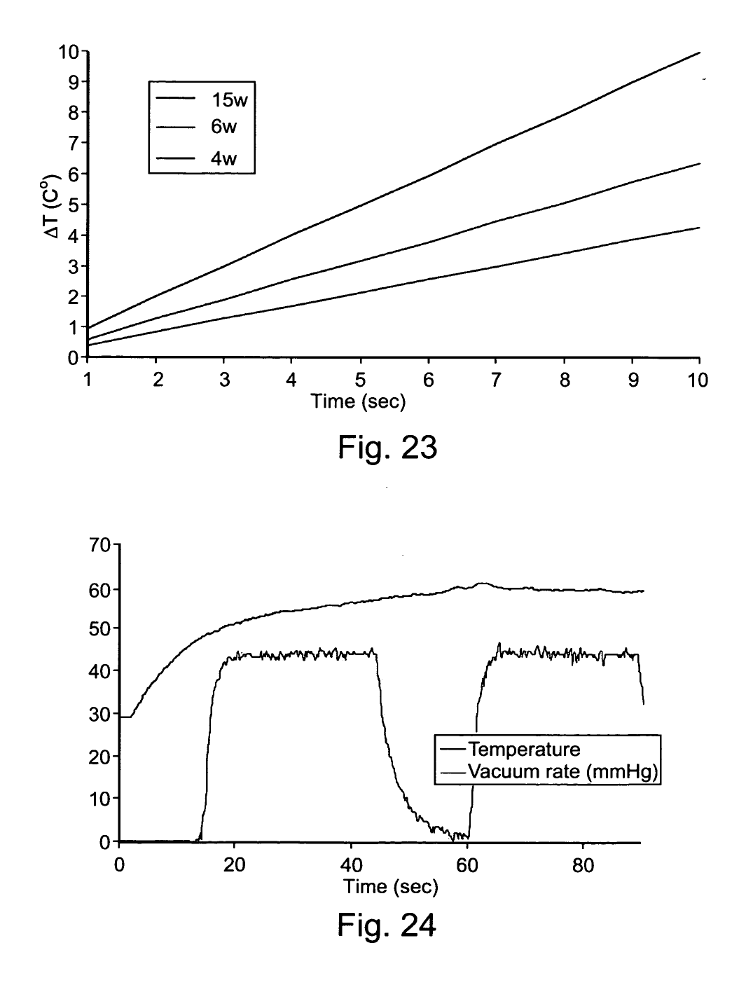

Reference is now made to FIG. 23, which is a graphical illustration of the effects of varying power on temperature changes. It is clear from these results that increased power leads to increased temperature changes.

3^{rd} Setup:

Results of various trials for more than 10 seconds are summarized in Table 4 below.

TABLE 4
Two types of thermal gradients in surrounding tissue, after 10 seconds;
distance between most and least heated points was 1.5 cm

| 10 sec | | | | |
|---|---|---|---|---|
| elapsed; neither | | | 6 watts_{Av} | 12 watts |
| airflow nor suction | 6 watts₁ₛₜ | 6 watts_{2nd} | (300 w/cm²) | (600 w/cm²) |
| ΔT_{1-11 sec} (° C.) | 6 | 5.5 | 5.75 | 13 |
| Max temp. after 10 | | | | |
| sec | | | | |
| Av. Slope (° C./Sec) | 0.6 | 0.55 | 0.575 | 1.3 |
| ΔT_{1.5 cm} (° C.) | 4.7 | 5.2 | 4.95 | 10.8 |
| (Max temp.) − (min. | | | | |
| temp.) in | | | | |
| surrounding tissue, | | | | |
| after 10 sec | | | | |

The two 6 watts-power trials resulted in different gradients, and thus were averaged. The average slope of the 12 watt-power was unreasonably higher than the one obtained in the 15 watt trial −1.3 vs. 1° C./Sec. Both cases, as well as others, which are not presented here, show poor repeatability of results. This is mainly reasoned by the need to replace or displace tissue sample after every irradiation, so that laser beam hits a fresh mucosa. However, another occurrence might have caused the lower gradient during the 15 watts test. The likely formation of jet in such a power could partially shift and blur the demarcated hotspot toward fiber entrance hole.

Damage to surrounding tissue: The maximal temperature of the upper layer of tissue did not exceed 46 degrees Celsius.

Control Via Central Control System:

The software-controlled setup monitors the maximal temperature all around the surrounding tissue and protects it from getting overheated. The maximal temperature is constantly analyzed from the IR images. The application automatically activates and regulates the CO₂ flow, the suction volume, the laser power or the pulse rate and duty cycle, to maintain the maximal temperature within a certain range. Regulation limits and increments are subjects to user definitions.

Since there was no evidence of thermal damage to surrounding tissue, the use of the “auto-protection” capability was meaningless in this experiment. However, the computerized setup is useful in outlining the principle relations between CO₂ gas flow, vacuum rate, time and maximal temperature of the surrounding tissue. The time intervals needed for substantial trend exploration varied from 40 to 100 seconds. In these periods, a mid-power laser beam will “drill” throughout the stomach wall. Therefore the power was constrained to 2 watts. By selecting continuous transmission mode, and by obtaining significant heat buildup via a previously noted fiber-heating phenomenon, it was possible to obtain significant heat build-up (above 46° C.).

Relations between parameters were investigated qualitatively and not quantitatively, as it is a multi-variable dependency and any numerical observation was relevant to the specific settings from which it was obtained.

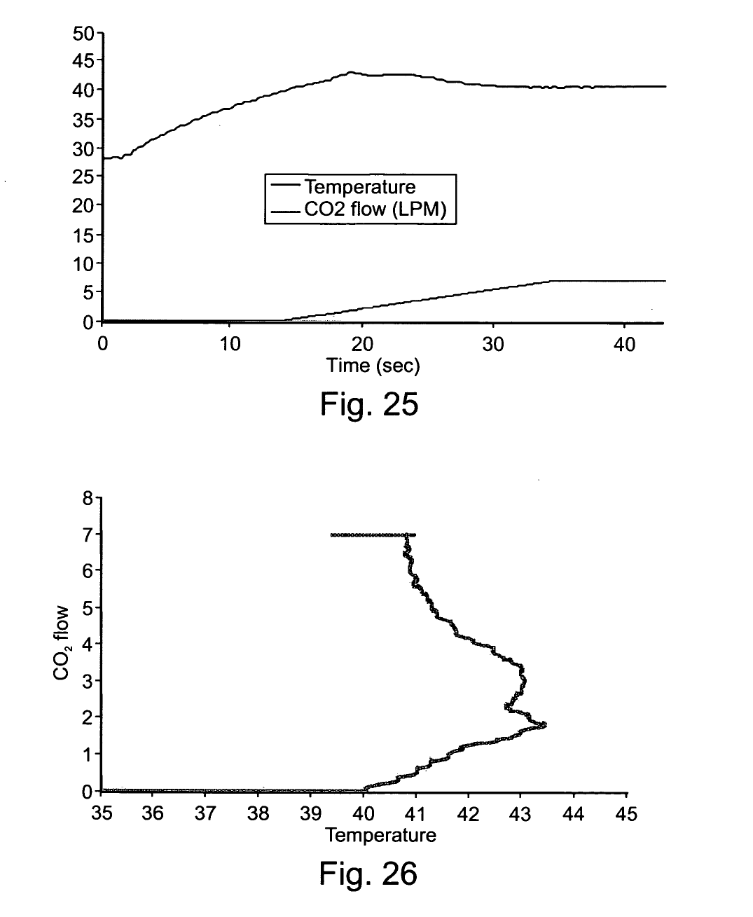

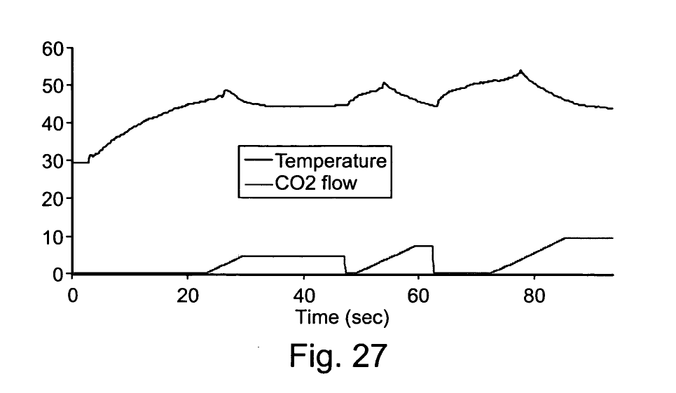

Results are shown in FIGS. 24-27, which are graphical illustrations of the effects of suction and CO₂ flow on temperature changes.

As shown in FIG. 24, maximum vacuum was set to 47 mmHg, “max temp allowed” was set to 46° C. (suction is activated when temp. exceeds 46° C.). The procedure was carried out at 2 watts under continuous wave administration.

Unlike previous experiments, suction had no considerable effect on the temperature. This phenomenon may be attributed to the following cause: the distance between fiber edge and irradiated tissue was very short, irradiation lasted 14 seconds, and air was not injected through the fiber. Under these conditions, it seems that even the maximal vacuum of 47 mmHg is not capable of evacuating the steam and preventing them from penetrating into the fiber. Consequently, the fiber overheats and heats the surrounding tissue (in practice it might even damage the endoscope). It was concluded that when the fiber edge is very close to the tissue and irradiation lasts more than a few seconds, even when using low power, airflow injection or higher vacuum must be used.

The CO₂ flow evidences major effects on the temperature. When CO₂ flow increases, the maximal temperature clearly declines down to a certain value, where it steadies. A higher flow limit enables dealing with higher temperature in shorter time intervals. There is, however, a major cause as to why the CO₂ has an effect while the suction does not. The CO₂ gas was compressed at 150 Atmospheres in a cylinder. When released into 1 Atm room pressure it cools significantly, then circulates within the cavity, and chills the fiber and tissue.

Another related issue is the absorption of 10.6 μm wavelength by the CO₂ gas. On one hand, the gas is heated and may heat the surrounding tissue. On the other hand, it attenuates the effective power that hits the tissue and causes the evaporation of steam. This compensative relation is thought to have a negligible effect since fiber edge and irradiated tissue were very close.

Summary of Experimentation:

Increasing the laser settings causes an increase in temperature changes, while increasing the suction and insufflation decreases the temperature changes. Furthermore, the distance and hitting angle, as well as the tissue characteristics, can have an effect as well. Thus, it is crucial to factor in all of these parameters during a procedure, and this may be accomplished during real time via a control system such as the one described in the present application.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for monitoring and control of a minimally invasive surgical procedure for a targeted tissue, the system comprising:
an endoscope, said endoscope comprises:
an input conduit for receiving an input for the targeted tissue; and
an output conduit for sending output from the targeted tissue;
an input mechanism for providing input to the targeted tissue through said input conduit of said endoscope;
a thermal output receiver for receiving thermal information from the targeted tissue through said output conduit of said endoscope; and
a processor in communication with said input mechanism and said thermal output receiver, said processor configured to receive said thermal output from said thermal output receiver and to adjust said input mechanism so as to adjust said input to the targeted tissue based on said thermal output, and wherein said receiving and adjusting is performable during a real-time procedure.

2. The system of claim 1, wherein said endoscope is selected from the group consisting of gastroscope, laparoscope, arthroscope, cystoscope, ureteroscope, pharyngoscope, bronchoscope and nephroscope.

3. The system of claim 1, wherein said input conduit comprises a laser beam conduit and wherein said input is a laser beam.

4. The system of claim 3, further comprising a flexible hollow waveguide placed within said input conduit, and wherein said flexible hollow waveguide is suitable for receiving an infrared laser beam therethrough.

5. The system of claim 1, wherein said input conduit comprises several input conduits.

6. The system of claim 5, wherein at least one of said several input conduits selected from the group consisting of a gas insufflation conduit, a suction conduit, a surgical tool conduit, and an illumination conduit.

7. The system of claim 1, wherein said output conduit is a viewing conduit.

8. The system of claim 7, wherein said viewing conduit is an infrared viewing conduit.

9. The system of claim 8, further comprising an infrared imaging fiber bundle, wherein said bundle is placed within said infrared viewing conduit for infrared thermal imaging of the tissue.

10. The system of claim 1, wherein said input mechanism is selected from the group consisting of a laser generator, a gas insufflator and a suction provider.

11. The system of claim 1, wherein said thermal output receiver is an infrared thermal output receiver.

12. The system of claim 11, wherein said infrared thermal output receiver is an infrared camera.

13. The system of claim 1, wherein said processor comprises a controller for controlling said input and based on said thermal output.

14. The system of claim 13, wherein said processor further comprises a thermal reader for reading said thermal output and incorporating said thermal output into a usable format.

15. The system of claim 14, wherein said usable format is a map.

16. The system of claim 14, wherein said usable format is a database.

17. A device for introduction of an infrared laser to a target tissue, the device comprising:
an endoscope having a conduit; and
a flexible hollow wave guide placed within said conduit, wherein said flexible hollow waveguide comprises: a hollow tube; a metal layer on the inner surface of the hollow tube; and a thin dielectric film of silver iodide over said metal layer.

18. The device of claim 17, wherein said endoscope is selected from the group consisting of gastroscope, laparoscope, arthroscope, cystoscope, ureteroscope, pharyngoscope, bronchoscope and nephroscope.

19. The device of claim 17, further comprising:
a infrared laser generator in communication with said endoscope for generating an infrared laser beam through said flexible hollow waveguide to the target tissue.

20. The device of claim 19, further comprising:
a feedback device in communication with the target tissue, said feedback device configured to receive output information from the target tissue upon application of said infrared laser beam.

21. The device of claim 20, wherein said feedback device is a thermal receiver and wherein said output information is thermal information from the target tissue.

22. The device of claim 21, wherein said thermal receiver comprises an infrared camera.

23. The device of claim 22, wherein said thermal receiver further comprises an infrared imaging fiber bundle in communication with said camera, said infrared imaging fiber bundle positioned through an additional conduit in said endoscope.

24. The device of claim 20, further comprising a processor in communication with said feedback device and said infrared laser generator, wherein said processor is configured to receive said output information from said feedback device and to adjust parameters of said infrared laser beam generated by said infrared laser generator based on said received output information.

25. A system for monitoring and control of thermal properties of tissue during a minimally invasive surgical procedure, the system comprising:
an endoscope, said endoscope comprises:
a laser delivery conduit having a flexible hollow waveguide for delivery of an infrared laser to the tissue; and
an imaging conduit having an infrared imaging fiber bundle placed within said imaging conduit for reading thermal properties of the tissue;
a laser generator for providing an infrared laser to the tissue through said laser delivery conduit of said endoscope;
an infrared camera for receiving thermal information from the targeted tissue through said imaging conduit of said endoscope; and
a processor in communication with said laser generator and said infrared camera, said processor configured to receive said thermal information from said infrared camera and to adjust said laser generator so as to adjust parameters of said laser provided to the tissue based on said thermal information, and wherein said receiving and adjusting is performable during a real-time procedure.

26. The system of claim 25, wherein said endoscope is selected from the group consisting of gastroscope, laparoscope, arthroscope, cystoscope, ureteroscope, pharyngoscope, bronchoscope and nephroscope.

27. The system of claim 25, further comprising a flexible hollow waveguide placed within said laser delivery conduit, and wherein said flexible hollow waveguide is suitable for receiving said infrared laser beam therethrough.

28. The system of claim 25, further comprising at least one additional input conduit.

29. The system of claim 28, wherein at least one of said additional input conduits is selected from the group consisting of a gas insufflation conduit, a suction conduit, a surgical tool conduit, and an illumination conduit.

30. The system of claim 25, wherein said processor comprises a controller for controlling said parameters of said laser based on said thermal information.

31. The system of claim 25, wherein said processor further comprises a thermal reader for reading said thermal information and incorporating said thermal information into a usable format.

32. The system of claim 31, wherein said usable format is a map.

33. The system of claim 31, wherein said usable format is a database.

34. A system for monitoring of minimally invasive surgery at a targeted site, the system-comprising:
a device for surgical contact with the targeted site, wherein the device comprises a distal end and a proximal end, said distal end configured to contact the targeted site and said proximal end accessible to a user, and an opening at said distal end connected to an opening at said proximal end;
an infrared imaging fiber bundle having a distal end and a proximal end, said infrared imaging fiber bundle positioned within said device such that said distal end of said infrared imaging fiber bundle is located at said opening of said distal end of said device, and said, proximal end of said infrared imaging fiber bundle is located at said opening of said proximal end of said device, wherein said distal end of said infrared imaging fiber bundle is configured to receive output information from said targeted site, and said proximal end of said infrared imaging fiber bundle is configured to send said output information to an output device; and
an output device in communication with said proximal end of said infrared imaging fiber bundle.

35. The system of claim 34, wherein said device is an endoscope.

36. The system of claim 34, wherein said output device is an infrared thermal imager.

37. The system of claim 34, further comprising a processor, said processor configured to receive said output information and to organize said output information into a readable format.

38. The system of claim 37, wherein said readable format is a color-coded map.

39. The system of claim 37, wherein said readable format is a report.

40. A method for monitoring and control of a minimally invasive surgical procedure, the method comprising:
introducing an infrared laser beam to a surgical site, said introducing including using adjustable parameters;
measuring thermal properties of said surgical site in response to said introducing;
processing said measured thermal properties; and
adjusting said adjustable parameters based on said processing,
wherein said adjusting is done during a time frame of the minimally invasive surgical procedure, and wherein said steps can be repeated as many times as necessary so as to achieve acceptable thermal properties.
